(19) United States
(12) Patent Application Publication
(10) Pub. No.: US 2025/0339036 A1
(43) Pub. Date: Nov. 6, 2025

(54) BLOOD PRESSURE EVALUATION WITH MACHINE LEARNING

(71) Applicant: Whoop, Inc., Boston, MA (US)

(72) Inventors: Rahul Kumar Krishnaiah Sevakula, Chestnut Hill, MA (US); Behnoosh Tavakoli, Needham, MA (US)

(21) Appl. No.: 19/080,402

(22) Filed: Mar. 14, 2025

Related U.S. Application Data
(60) Provisional application No. 63/641,196, filed on May 1, 2024.

(51) Int. Cl. A61B 5/021; A61B 5/00; A61B 5/024; G06N 3/0442; G16H 50/20

(52) U.S. Cl. CPC A61B 5/02116; A61B 5/02438; A61B 5/6802; G06N 3/0442; G16H 50/20; A61B 2562/164

(57) ABSTRACT

A method for baseline blood pressure estimation of a user of a wearable physiological monitor. The method comprising identifying a segment of pulse data related to cardiac activity of the user during a portion of a sleep session, wherein the segment of pulse data is obtained by the wearable physiological monitor; determining, from the segment of pulse data, a resting heart rate value of the user during the portion of the sleep session; identifying a machine learning model trained to receive as input one or more features including an input resting heart rate value obtained during a first time period and predict an indicator of blood pressure during a second time period; and providing the resting heart rate value to the machine learning model to obtain an indicator of baseline blood pressure for the user.

### RELATED APPLICATIONS

This application claims priority to U.S. Prov. App. No. 63/641,196 filed on May 1, 2024, the entire content of which is hereby incorporated by reference.

### TECHNICAL FIELD

The present disclosure generally relates to wearable physiological monitoring systems, and more specifically to estimating a blood pressure metric based on signals from a wearable physiological monitoring device.

### BACKGROUND

Physiological monitoring systems can monitor heart rate activity via sensors such as photoplethysmography (PPG) sensors or electrocardiogram (ECG) sensors, and use this data to provide metrics for sleep performance, activity, strain, recovery, and so forth. While a variety of derived and related physiological metrics such as pulse oxygenation and respiration rate can be derived from this data, there remains a need for improved blood pressure estimation using physiological monitoring data.

### SUMMARY

According to an aspect of the present disclosure there is provided a method for baseline blood pressure estimation of a user of a wearable physiological monitor. The method comprises identifying a segment of pulse data related to cardiac activity of the user during a portion of a sleep session, wherein the segment of pulse data is obtained by the wearable physiological monitor; determining, from the segment of pulse data, a resting heart rate value of the user during the portion of the sleep session; identifying a machine learning model trained to receive as input one or more features including an input resting heart rate value obtained during a first time period and predict an indicator of blood pressure during a second time period; and providing the resting heart rate value to the machine learning model to obtain an indicator of baseline blood pressure for the user.

According to a further aspect of the present disclosure there is provided a non-transitory computer readable medium comprising instructions that, when executed by a processor of a computing device, cause the processor to identify a plurality of segments of pulse data related to cardiac activity of a user during a plurality of portions of a sleep session, wherein the plurality of segments of pulse data are obtained by a wearable physiological monitor; determine, from the plurality of segments of pulse data, a resting heart rate value of the user the sleep session; identify a machine learning model trained to receive as input one or more features including an input resting heart rate value obtained during a first time period and predict an indicator of blood pressure during a second time period; and provide the resting heart rate value to the machine learning model to obtain an indicator of baseline blood pressure for the user.

According to an additional aspect of the present disclosure, there is provided a system comprising a wearable physiological monitor including a photoplethysmography (PPG) sensor, a first processor configured to obtain pulse data for a user based on a signal from the PPG sensor, and a communications interface for coupling with a remote resource; a computing device coupled in a communicating relationship with the wearable physiological monitor, the computing device including a second processor configured by computer executable code to: identify a plurality of segments of pulse data related to cardiac activity of the user across a plurality of sleep sessions, wherein the plurality of segments of pulse data are obtained by the wearable physiological monitor; determine, from the plurality of segments of pulse data, a resting heart rate value of the user across the plurality of sleep sessions; identify a machine learning model trained to receive as input one or more features including an input resting heart rate value obtained during a first time period and predict an indicator of blood pressure during a second time period; and provide the resting heart rate value to the machine learning model to obtain an indicator of baseline blood pressure for the user.

According to another aspect of the present disclosure there is provided a method for baseline blood pressure estimation comprising: storing demographic information for a user; storing a segment of pulse data from a wearable physiological monitor worn by the user, the segment of pulse data related to cardiac activity of the user during a sleep session; determining, from the segment of pulse data, a resting heart rate value of the user during a predetermined portion of the sleep session; providing a machine learning model trained to generate an indicator of baseline blood pressure for the user in response to at least the resting heart rate value of the user and the demographic information for the user; and providing the resting heart rate value and the demographic information to the machine learning model to obtain the indicator of baseline blood pressure for the user

According to a further aspect of the present disclosure there is provided a method comprising receiving, from a wearable physiological monitor worn by a user, pulse data including a plurality of heart pulse samples of the user during a sleep session; extracting a portion of the pulse data corresponding to an initial period of the sleep session; fitting a model to the portion of the pulse data, wherein the model encodes dynamics of the portion of the pulse data during the initial period of the sleep session; and calculating a blood pressure indicator score for the user based on the model fit to the portion of the pulse data.

According to another aspect of the present disclosure there is provided a non-transitory computer readable medium comprising instructions that, when executed by a processor of a computing device, cause the processor to: receive, from a wearable physiological monitor worn by a user, pulse data including a plurality of heart pulse samples of the user during a sleep session; extract a portion of the pulse data corresponding to an initial period of the sleep session; fit a model to the portion of the pulse data, wherein the model encodes changes in dynamics of the portion of the pulse data during the initial period of the sleep session; and calculate a blood pressure indicator score for the user based on the model fit to the portion of the pulse data.

According to another aspect of the present disclosure there is provided a system comprising: a wearable physiological monitor including a photoplethysmography (PPG) sensor, a first processor configured to obtain pulse data for a user based on a signal from the PPG sensor, and a communications interface for coupling with a remote resource; a computing device coupled in a communicating relationship with the wearable physiological monitor, the computing device including a second processor configured by computer executable code to: receive, from a wearable physiological monitor worn by a user, pulse data including a plurality of heart pulse samples of the user during a sleep session; extract a portion of the pulse data corresponding to an initial period of the sleep session; fit a dynamics model to the portion of the pulse data, wherein the dynamics model encodes changes in heart rate during the initial period of the sleep session; and calculate a blood pressure indicator score for the user based on the dynamics model fit to the portion of the pulse data.

According to an additional aspect of the present disclosure there is provided a computer program product comprising executable code embodied in a non-transitory computer readable medium that, when executing on one or more computing devices, performs the steps of: receiving, from a wearable physiological monitor worn by a user, accelerometer data indicative of movement of the wearable physiological monitor during a time window, the time window corresponding to a portion of a sleep session of the user; identifying, based on the accelerometer data, a principal direction of movement of the wearable physiological monitor during the time window; generating a first waveform from the accelerometer data, the first waveform comprising data indicative of movement of the wearable physiological monitor along the principal direction of movement; and calculating a plurality of respiratory onsets for the user during the time window based on a plurality of local extrema of the first waveform.

According to a further aspect of the present disclosure there is provided a method comprising: receiving, from a wearable physiological monitor worn by a user, accelerometer data indicative of movement of the wearable physiological monitor during a time window, the time window corresponding to a portion of a sleep session of the user; identifying, based on the accelerometer data, a principal direction of movement of the wearable physiological monitor during the time window; generating a first waveform from the accelerometer data, the first waveform comprising data indicative of movement of the wearable physiological monitor along the principal direction of movement; and calculating a plurality of respiratory onsets for the user during the time window based on a plurality of local extrema of the first waveform.

According to another aspect of the present disclosure there is provided a system comprising: a wearable physiological monitor including a photoplethysmography (PPG) sensor, a first processor configured to obtain pulse data for a user based on a signal from the PPG sensor, an accelerometer, and a communications interface for coupling with a remote resource; a computing device coupled in a communicating relationship with the wearable physiological monitor, the computing device including a second processor configured by computer executable code to: receive, from a wearable physiological monitor worn by a user, accelerometer data indicative of movement of the wearable physiological monitor during a time window, the time window corresponding to a portion of a sleep session of the user; identify, based on the accelerometer data, a principal direction of movement of the wearable physiological monitor during the time window; generate a first waveform from the accelerometer data, the first waveform comprising data indicative of movement of the wearable physiological monitor along the principal direction of movement; and calculate a plurality of respiratory onsets for the user during the time window based on a plurality of local extrema of the first waveform.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1A is a flow chart illustrating a method for baseline blood pressure estimation.

FIG. 1B is a flow chart illustrating further steps of the method shown in FIG. 1A.

FIG. 2A is a flow chart illustrating a method for baseline blood pressure estimation.

FIG. 2B is a flow chart illustrating a method for aggregated baseline blood pressure estimation.

FIG. 3A shows portions of a system for baseline blood pressure estimation.

FIG. 3B shows portions of a system for baseline blood pressure estimation.

FIG. 3C shows a portion of a system for aggregated baseline blood pressure estimation.

FIG. 4 illustrates feature points extracted from a pulse of a segment of pulse data.

FIG. 5 shows an encoder-decoder network.

FIG. 6 illustrates synthetic pulses for training an encoder-decoder network.

FIG. 7 is a flow chart illustrating a method for calculating a blood pressure indicator score based on pulse data.

FIG. 8A shows an illustrative example of hypertension classification using the method 700 of FIG. 7.

FIG. 8B shows an illustrative example of hypertension classification using the method 700 of FIG. 7.

FIG. 8C shows an illustrative example of hypertension classification using the method 700 of FIG. 7.

FIG. 8D shows an illustrative example of hypertension classification using the method 700 of FIG. 7.

FIG. 8E shows an illustrative example of hypertension classification using the method 700 of FIG. 7.

FIG. 8F shows an illustrative example of hypertension classification using the method 700 of FIG. 7.

FIG. 9A is a flow chart illustrating a method for calculating respiratory onsets of a user.

FIG. 9B is a flow chart illustrating further steps of the method shown in FIG. 9A.

FIG. 9C is a flow chart illustrating further steps of the method shown in FIGS. 9A and 9B.

FIG. 10A illustrates accelerometer based respiratory onset detection.

FIG. 10B illustrates accelerometer based respiratory onset detection.

FIG. 10C illustrates accelerometer based respiratory onset detection.

FIG. 11 shows a physiological monitoring device.

FIG. 12 illustrates a physiological monitoring system.

FIG. 13 shows a smart garment system.

FIG. 14 is a block diagram of a computing device.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

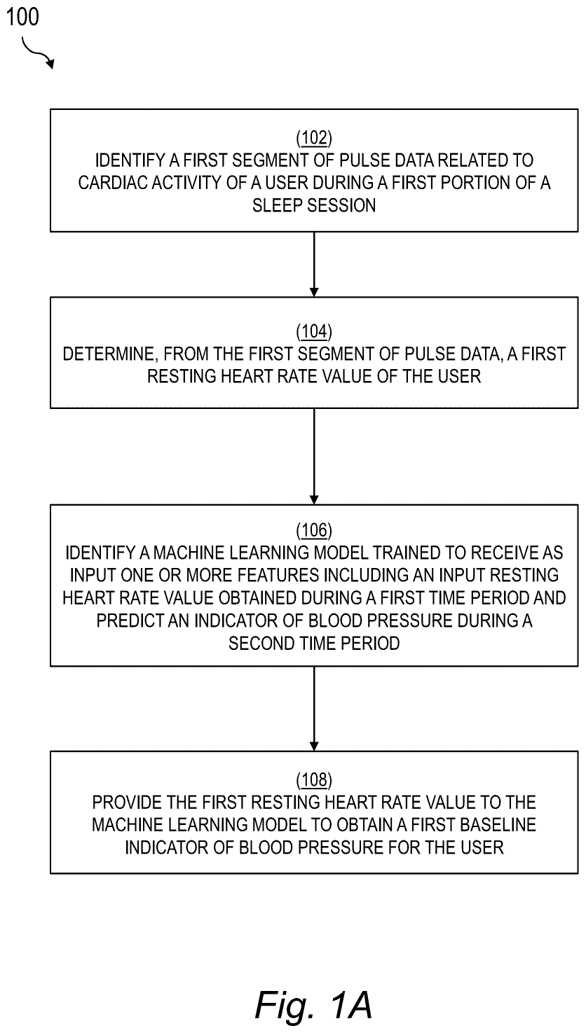

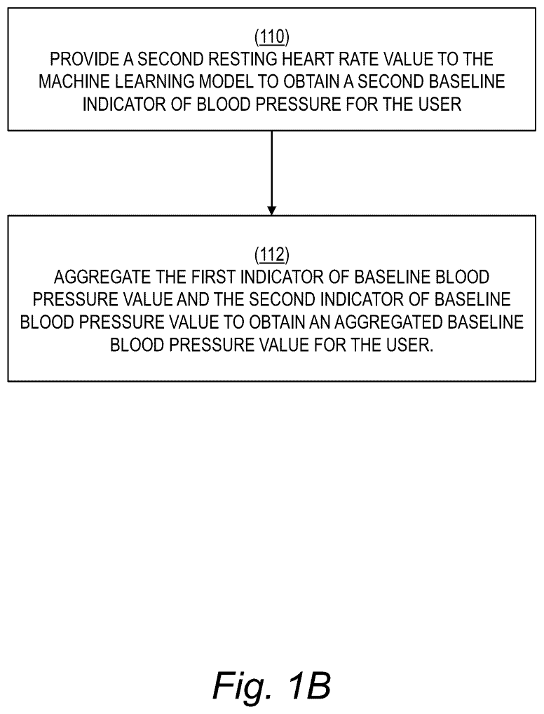

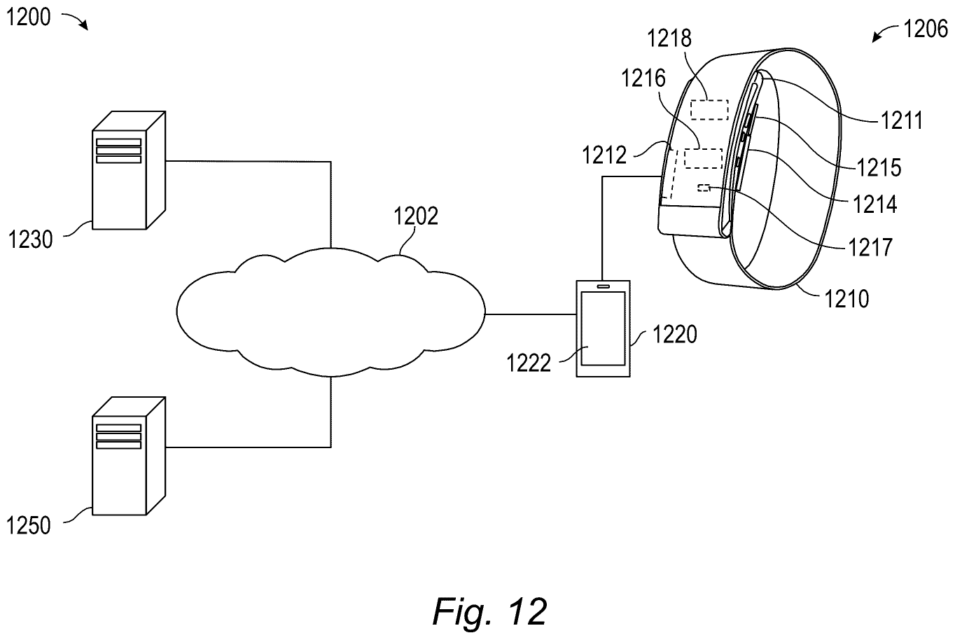

FIG. 1A is a flow chart illustrating a method 100 for baseline blood pressure estimation of a user of a wearable physiological monitor. The method 100 may be used in cooperation with any of the devices, systems, and methods described herein, such as by a user device (e.g., a mobile device) that is communicatively coupled to a wearable, continuous physiological monitoring device. For example, the method 100 may be used with the one or more user devices 1220 that are communicatively coupled to the physiological monitor 1206, as illustrated in FIG. 12. In general, the method 100 determines a resting heart rate value for a user during a portion of a sleep session and predicts an indicator of baseline blood pressure for the user using the resting heart rate value. As such, the method 100 provides an efficient and non-invasive approach for predicting an indicator of baseline blood pressure for a user—either a baseline blood pressure value (e.g., a baseline systolic or diastolic blood pressure value) or a hypertensive classification score (e.g., a systolic or diastolic hypertension classification)—from pulse data obtained from the user while the user is at rest. Obtaining the pulse data from the user while the user is asleep helps improve the accuracy of the blood pressure estimation by obtaining clean pulse data. Obtaining pulse data when a user is asleep results in a cleaner pulse signal as the heart rate is less likely to fluctuate than when they are awake, and external factors and noise are less likely to affect the sensor readings.

As shown in step 102, the method 100 may include identifying a first segment of pulse data related to cardiac activity of a user during a first portion of a sleep session. The first segment of pulse data is obtained by a wearable physiological monitor. For example, the first segment of pulse data may be obtained from a physiological monitor such as the physiological monitor 1206 in FIG. 12 by a user device such as the one or more user devices 1220. More particularly, the first sequence of pulse data may be obtained from a suitable sensor (e.g., a photoplethysmogram (PPG) sensor) coupled to the physiological monitor.

The first segment of pulse data comprises a sequence of pulses (e.g., heart beats) that relate to the cardiac activity of the user while the user sleeps. That is, the first segment of pulse data corresponds to a waveform or time series of values characterizing the cardiac activity of the user during a first portion of a sleep session of the user. The first portion of the sleep session corresponds to a subframe or time period of the sleep session (e.g., a 20 second portion, 30 second portion, 40 second portion, etc.). As such, the number of pulses within the first segment of pulse data depends on the length of the first portion of the sleep session. In one embodiment, the first portion of the sleep session is chosen such that the first segment of pulse data comprises in the range of 20 to 40 pulses.

The first segment of pulse data may comprise pulses that occur during a single respiratory cycle of the user. Here, a respiratory cycle (or breathing cycle) may be considered as spanning a time frame within which the user performs a single inhalation (inspiration) and a subsequent single exhalation (expiration). As described herein, a single respiratory cycle may be identified using accelerometer data of the wearable physiological monitor during the first portion of the sleep session. A respiratory cycle may also or instead be identified using other techniques, such as by using respiratory sinus arrhythmia—heart rate fluctuations associated with respiration—to detect inhalation and exhalation based on an associated increasing and decreasing heart rate. As such, instead of analyzing a fixed time frame of data to estimate a blood pressure indicator (e.g., a fixed time window of 20 seconds, 30 seconds, etc.), one or more time frames that are aligned to the user's respiratory cycles are analyzed. This may enable more physiologically relevant data to be used to estimate a blood pressure indicator.

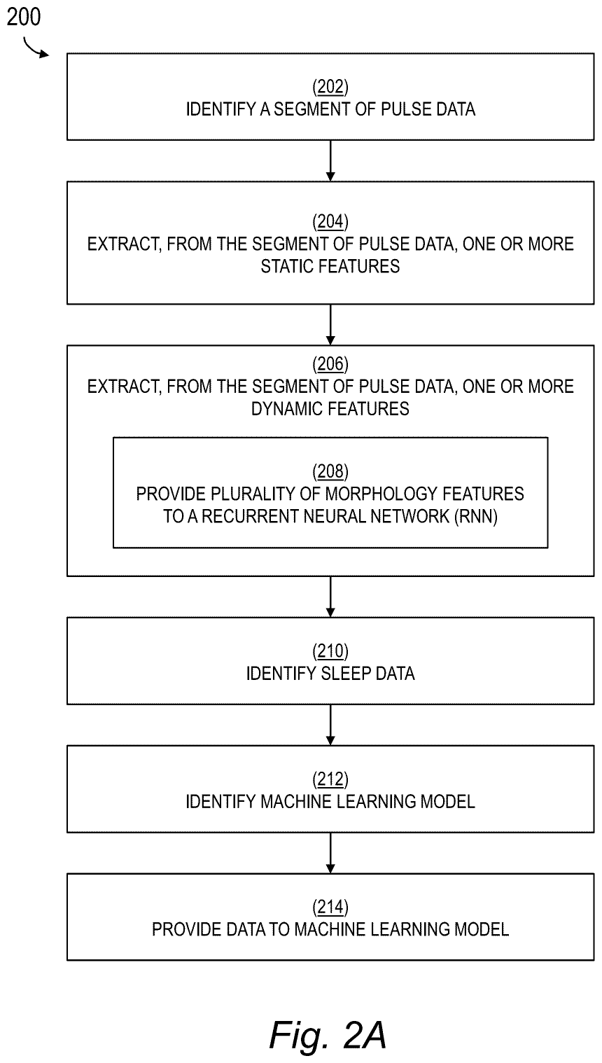

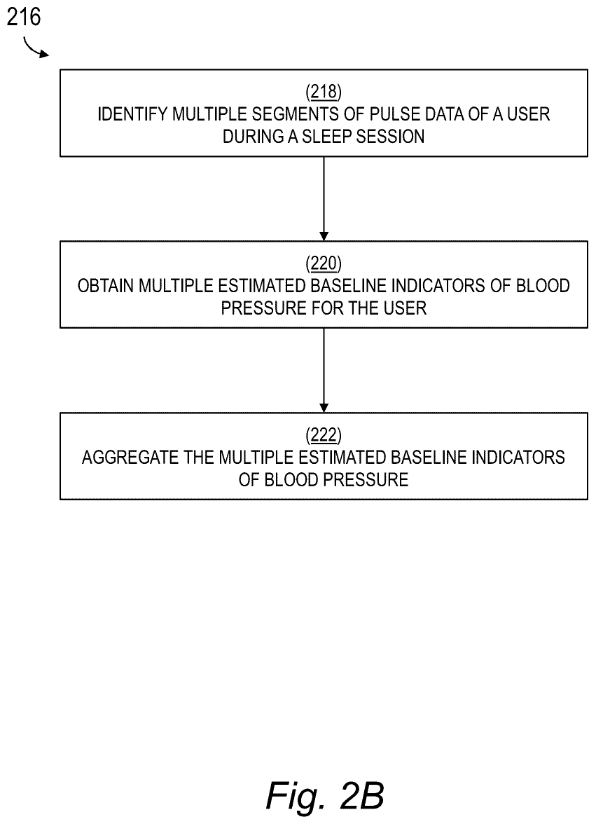

In one embodiment, and as described in more detail in relation to FIG. 2A, the method 100 may include processing the first segment of pulse data. For example, the first segment of pulse data may be normalized or otherwise transformed. As a further example, pulses within the first segment of pulse data may be filtered or selected such that pulses that satisfy one or more quality criteria are maintained for further processing (e.g., pulses that have a well-defined dicrotic notch).

In one embodiment, cuff calibration can be performed using manual or automatic measurements from a blood pressure (BP) cuff, which can provide a check of the accuracy of pulse data obtained from the PPG sensor of the wearable physiological monitor. This may include using the BP cuff measurements to calibrate the PPG sensor at regular intervals, while relying on the optical data from the PPG sensor for continuous monitoring between these calibration points. Initially, the BP cuff may be used to measure the user's blood pressure. This measurement provides a reference point for the PPG sensor. In general, a BP cuff may inflate and deflate to apply a range of pressure to the user's arm, typically starting at a value above a representative systolic pressure and then ranging to a value below a representative diastolic pressure. At the same time, pulse activity may be monitored in the underlying vasculature, e.g., manually, or with an acoustic or pressure sensor, which provides an indication of the pressure at which a heart pumps (systolic) and relaxes (diastolic) during cardiac activity. These values are recorded in units such as millimeters of mercury (mmHg) and serve as a benchmark for the PPG sensor's readings. Once the BP cuff has obtained accurate blood pressure measurements, e.g., using a suitable clinical protocol, the PPG sensor may be calibrated against these values. The PPG sensor can then be adjusted on a prospective basis to better align PPG-based measurements with BP cuff measurements. This calibration process involves comparing the pulse data from the PPG sensor with the blood pressure data from the BP cuff and making necessary adjustments to the PPG sensor's algorithm to account for the calibration difference. The calibration can be performed on a per-user basis (e.g., each user may calibrate their own wearable physiological monitor using data obtained from a BP cuff). Also, or instead, calibration data can be performed centrally for a type of physiological monitor sensor, or for a population or demographic sub-group, and rolled out to corresponding wearable physiological monitors. As such, a BP cuff may be used for calibrating particular optical measurements, and optical data may be used for monitoring blood pressure between calibrations.

As shown in step 104, the method 100 may include determining, from the first segment of pulse data, a first resting heart rate value of the user during the first portion of the sleep session.

Resting heart rate corresponds to the rate at which a heart is pumping when the body is at rest. The resting rate value of a user typically corresponds to the point at which the user's heart is pumping the least amount of blood to supply oxygen to the body. Most healthy adults have a resting heart rate in a range of 55 to 85 beats per minute (bpm). However, numerous factors can affect resting heart rate such as stress, hormones, medication, physical activity level, and the like. Therefore, obtaining a heart rate value for a user during the day will likely lead to a noisy or inaccurate estimation of the user's resting heart rate. Obtaining the heart rate of the user while the user is at sleep allows the effect of these factors to be reduced and thus provides a more accurate indication of the resting heart rate.

The first resting heart rate value is a number indicating the heart rate of the user during the first portion of the sleep session. Typically, a resting heart rate value is measured in beats per minute (bpm). The first resting heart rate value can be determined from the first segment of pulse data by dividing the number of pulses within the first segment of pulse data by the length (in minutes) of the first segment of pulse data (e.g., 30 pulses within a segment of pulse data having a length of 0.5 minutes corresponds to a heart rate value of 60). A pulse or beat counting algorithm is used to calculate the number of pulses within the first segment of pulse data. In an example, a peak finding algorithm is used to identify the number of peaks within the first segment of pulse data, where each peak represents a single pulse. Advantageously, obtaining the first segment of pulse data while the user is asleep helps reduce the amount of noise that is typically present in the pulse morphology while the user is active thereby helping to improve the accuracy of the estimated number of pulses and resulting resting heart rate value.

A variety of techniques may be used to ensure that cardiac data is measured consistently, e.g., during a predetermined portion, interval, or window of a sleep session, from day to day, so that historical data accurately reflects non-transient physiological changes, and so that suitable inferences can be drawn, e.g., by machine learning models or other analytical tools, based on new measurements. For example, cardiac measurements captured during deep sleep are typically consistent for a user, and permit identification of significant changes in health, rest, strain, and so forth. Other stages may also be effective for the purposes described herein, such as REM stage with or without incorporating the time from falling asleep as a model feature. In one aspect, the method 100 may include identifying a particular stage of sleep (e.g., deep sleep, slow wave sleep, REM sleep, light sleep), as well as transitional timing such as the amount of time elapsed after falling asleep, or the amount of time prior to waking. This data may be used to select the predetermined portion of a sleep session for acquiring training data, and for acquiring a new measurement when the trained model is applied to make inferences about a baseline blood pressure for a user. Thus, for example a physiological signal such as a PPG signal, ECG signal, or other cardiac signal or the like may be measured during the predetermined portion of a sleep interval when acquiring data to evaluate an indicator of baseline blood pressure for a user.

In another aspect, after a suitable portion of the sleep session is identified, the acquired signal may be further processed, e.g., as described herein, to extract features such as a heart rate, a heart rate variability, or any of the other cardiac signal features described herein, or any other suitable features for a physiological signal of interest. for example, measuring the resting heart rate may include capturing a physiological signal during a particular stage of sleep, such as the REM sleep stage, and calculating heart rate metrics based on the physiological signal such as an average or median of a heart rate or heart rate variability for these measurements. In another aspect, the heart rate metric(s) may include a weighted average of heart rate values that more heavily weights measurements captured near the end of a stage of sleep. Stages of sleep are cyclical, with multiple episodes of each stage typically occurring during a night. Thus, measurements may also or instead be taken for all episodes of a particular stage, or for a last or most recent complete stage before waking, or based on a time from falling asleep. In another aspect, other heart rate metrics or features may be extracted from the physiological signal acquired over the predetermined portion of the sleep session, such as a pulse shape, a pulse width, a pulse slope, a pulse height, a pulse amplitude, and so forth. Other metrics indicative of quality may also or instead be calculated, and used as a weighting or filtering mechanism for the data in the physiological signal. More generally, any suitable techniques for characterizing, averaging, filtering, windowing, or weighting physiological measurements such as cardiac data may usefully be employed in this context to obtain one or more descriptive metrics for the physiological signal during the sleep session.

As shown in step 106, the method 100 may include identifying a machine learning model trained to receive as input one or more features including an input resting heart rate value obtained during a first time period and predict an indicator of blood pressure during a second time period.

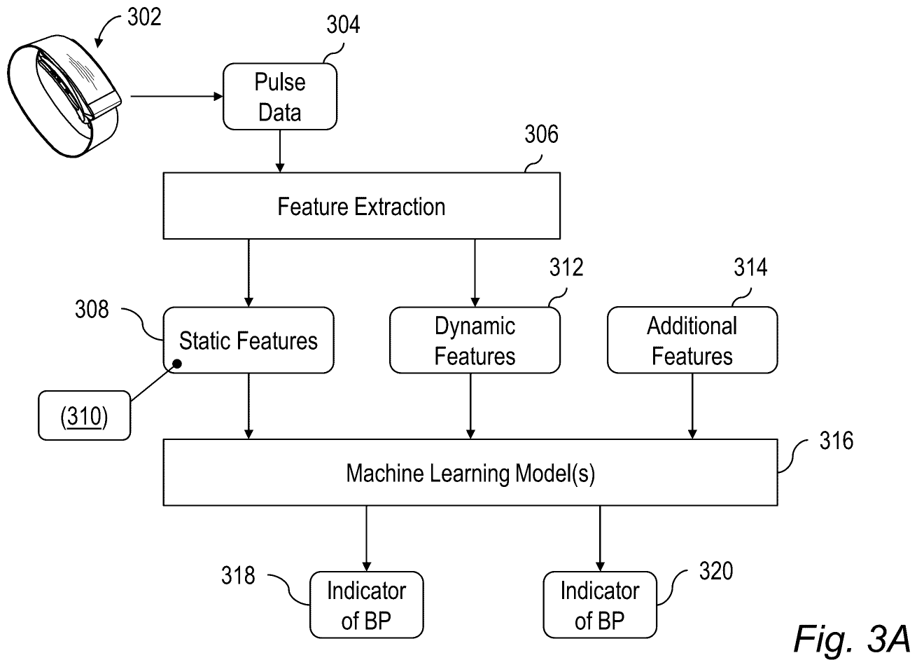

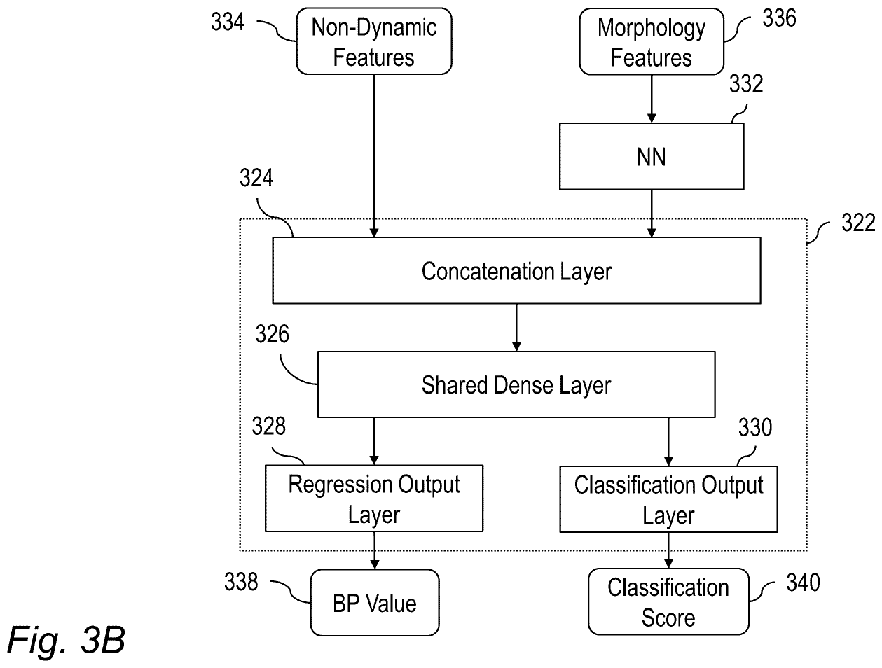

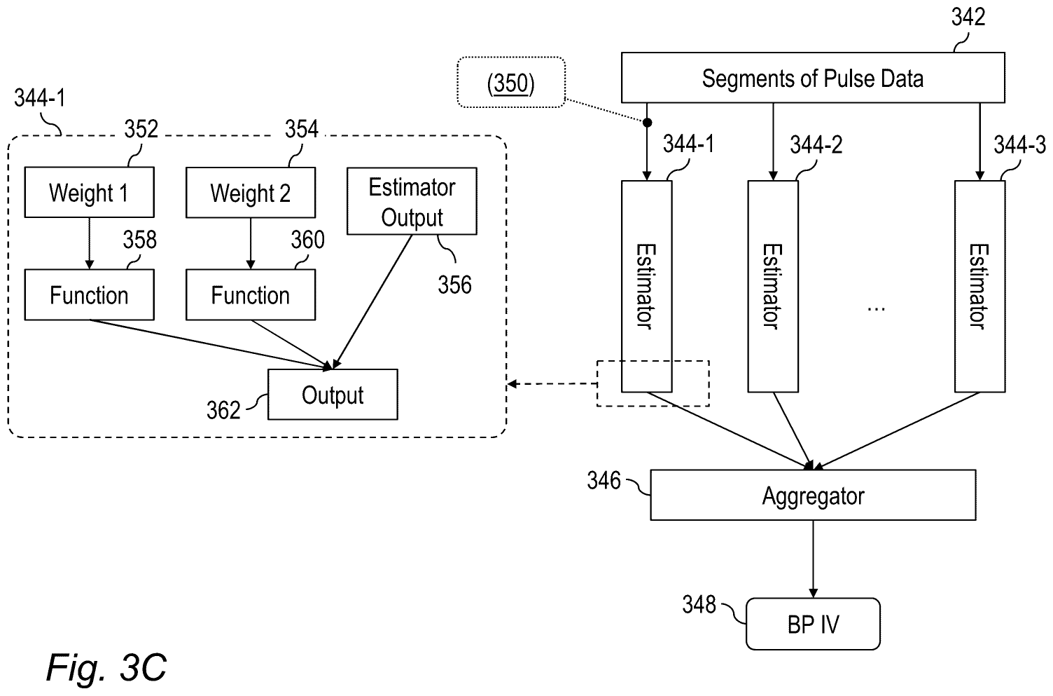

The machine learning model can be any suitable machine learning algorithm or model. The choice of machine learning model depends on the indicator of blood pressure being predicted. That is, if a baseline blood pressure value is being predicted as the indicator of blood pressure, then a regression algorithm is used; whereas if a hypertension classification score is being predicted, then a classification algorithm is used. Examples of suitable regression algorithms include linear regression, support vector regression, Bayesian linear regression, and artificial neural networks. Examples of suitable classification algorithms include support vector machines (SVMs), naïve Bayes classifiers, and artificial neural networks. In one embodiment, the machine learning model is an artificial neural network comprising an input layer, at least one hidden layer, and at least one output layer (as described in more detail below in relation to FIGS. 3A and 3B). The at least one output layer includes an output layer for predicting a baseline blood pressure value and/or an output layer for predicting a hypertension classification score. In general, the machine learning model may provide an inference based on demographic information and a single resting heart rate measurement for a user, or based on a time series of resting heart rate measurements, e.g., over a number of days, a week, a month, or some other suitable interval.

In one embodiment, and as described in more detail below in relation to FIG. 2A, the machine learning model is operable to receive additional features to predict the baseline indicator. The additional features can include static features that characterize average pulse morphology during the portion of the sleep session, dynamic features that characterize temporal variation in pulse morphology during the portion of the sleep session, pulse arrival time features, demographic features, and sleep data.

The method 100 may include, prior to step 108, the step of training the machine learning model on a training data set as described below. The skilled person will appreciate that the machine learning model can be trained in any suitable manner using a training approach suitable for the machine learning model or algorithm used. Further details regarding training of an artificial neural network are provided in relation to FIGS. 3A and 3B below.

As shown in step 108, the method 100 may include providing the first resting heart rate value to the machine learning model to obtain a first indicator of baseline blood pressure for the user.

The first indicator of baseline blood pressure may be a baseline blood pressure value for the user. The baseline blood pressure value is one of a baseline systolic blood pressure value or a baseline diastolic blood pressure value. In one embodiment, and as described in more detail below in relation to FIG. 2A, one or more trends in baseline blood pressure for the user may be tracked based on the baseline blood pressure value and one or more historical blood pressure values of the user. That is, blood pressure values for the user may be sequentially obtained over a period of time (e.g., 5 days, 7 days, 14 days, 1 month, 2 months, 6 months, etc.) and trends or changes in the user's blood pressure tracked and identified.

The first indicator of baseline blood pressure may be a hypertensive classification score for the user. The hypertensive classification score provides a probability of the user being hypertensive. The hypertensive classification score is one of a systolic hypertension classification score or a diastolic hypertension classification score.

FIG. 1B is a flow chart illustrating further steps that may be performed as part of the method 100 shown in FIG. 1A. That is, the steps shown in FIG. 1B may be performed after step 108 of the method 100 in the ordered sequentially numbered, or before or concurrently with any of the other steps in FIG. 1A. The additional steps shown in FIG. 1B allow for an indicator of baseline blood pressure for the user to be estimated from multiple segments of pulse data obtained during the same sleep session (as described in relation to FIG. 3C below). That is, rather than estimate the indicator of baseline blood pressure for the user from a single segment of pulse data, multiple estimates from across multiple segments of pulse data are aggregated to determine a robust, comparable, and accurate indicator of blood pressure for the user. While the following description relates primarily to aggregating two baseline indicators of blood pressure, the skilled person will appreciate that the method is not intended to be limited as such and may be expanded to an aggregation of more than two baseline indicators of blood pressure (e.g., 5 segments and 5 baseline indicators, 10 segments and 10 baseline indicators, 100 segments and 100 baseline indicators, 300 segments and 300 baseline indicators, etc.).

As shown in step 110, the method 100 may include providing a second resting heart rate value to the machine learning model to obtain a second indicator of baseline blood pressure for the user. The second resting heart rate value may be determined from a second segment of pulse data related to cardiac activity of the user during a second portion of the sleep session. As such, at step 110 a second (or further) segment of pulse data during a second (or further) portion of the sleep session may be obtained and a resting heart rate value determined for the second segment of pulse data (as described for the first segment of pulse data in relation to step 104 above). The resting heart rate value may then be provided to the machine learning model to determine a second indicator of baseline blood pressure. The skilled person will appreciate that the description of the machine learning model provided above for the first resting heart rate value in relation to step 106 is applicable to the step 110 for the second resting heart rate value.

As shown in step 112, the method 100 may include aggregating the first indicator of baseline blood pressure value and the second indicator of baseline blood pressure value to obtain an aggregated indicator of baseline blood pressure value for the user.

The first indicator of baseline blood pressure value and the second indicator of baseline blood pressure value may be aggregated by taking an average (e.g., mean) of the two indicator values. Similarly, if more than two indicators of baseline blood pressure values are being aggregated, then the average (e.g., mean, median, mode) across all indicators may be calculated to determine the aggregated indicator of baseline blood pressure value for the user. Alternatively, the first indicator of baseline blood pressure value and the second indicator of baseline blood pressure value may be aggregated using a weighted aggregation strategy comprising one or more weighting rules. In general, the weighted aggregation strategy may use any suitable weighting rules to combine estimated indicators of baseline blood pressure values based on morphological and temporal characteristics of the pulse data used to determine the estimated indicators of baseline blood pressure values (as shown in FIG. 3C described below). Advantageously, a weighted aggregation strategy provides a heuristic approach to aggregation whereby greater weight is applied to indicators of blood pressure values which are more likely to have been generated from physiologically useful and high-quality pulse data. The weighted aggregation strategy provides an efficient and effective approach for transforming and combining multiple indicators of baseline blood pressure into a single accurate and robust value which can then be used for various downstream tasks such as identifying longitudinal trends in a user's baseline blood pressure.

In general, a weighting rule determines a weight wⱼ that can be applied to an indicator of baseline blood pressure value bpⱼ such that the aggregated indicator of baseline blood pressure value for K segments of pulse data may be calculated as

$${Σ}_{j = 1}^{K} {w}_{j} b {p}_{j} .$$

Multiple weighting rules,

$${w}_{j}^{( 1 )} , {w}_{j}^{( 2 )} ,$$

etc. may be combined and the aggregated indicator of baseline blood pressure value for L weighting rules may be calculated as

$${Σ}_{j = 1}^{K} ( {Σ}_{i = 1}^{L} {w}_{j}^{( i )} ) b {p}_{j} .$$

As described in more detail below, functions may be used to attenuate and/or control individual weighting rules, and the weights may be normalized in any suitable manner to weight individual indicators based on objective indicia of reliability.

The one or more weighting rules may comprise a first weighting rule that is based on the deviation of the heart rate of the pulse data from a resting heart rate value. That is, a segment of pulse data that has an average heart rate that is closer to the resting heart rate of the user during the sleep session may be more useful than a segment of pulse data whose average heart rate deviates more from the resting heart rate of the user during the sleep session. Here, a useful segment of pulse data may indicate that the indicator of baseline blood pressure value generated from the segment of pulse data should contribute more to the aggregated indicator of baseline blood pressure value than a less useful segment of pulse data. The weighting applied by the first weighting rule, for an indicator of baseline blood pressure generated from a segment of pulse data, may be calculated by identifying the absolute difference between the average (mean) heart rate of the segment of pulse data and the overall resting heart rate of the user (determined from across the sleep session). As such, a first weighting rule assigns a greater weight to the first indicator of baseline blood pressure value than to the second indicator of baseline blood pressure value when a first difference between the first resting heart rate value and a resting heart rate value of the sleep session is less than a second difference between the second resting heart rate value and the resting heart rate value of the sleep session. Any suitable approach may be used to determine the resting heart rate of the user during the sleep session. In on example, the resting heart rate of the user during the sleep session may be determined by identifying a period of time in which the user was in a deep sleep state prior to waking up (e.g., the final deep sleep state of the user during the sleep session) and then extracting a segment of pulse data (e.g., 30 second segment, 60 second segment, etc.) during this period of time and calculating the average heart rate for the segment of pulse data.

The first weighting rule may be represented mathematically as follows. Given K segments of pulse data, let the segment index be j and the difference between the segment's mean heart rate and the resting heart rate of the sleep session be hrdⱼ. The weight assigned to the segment, wⱼ, may be calculated as

$${w}_{j} = \frac{exp ( - α \cdot {hrd}_{j} )}{{Σ}_{j = 1}^{K} {w}_{j}} .$$

The parameter α determines the decay of weights as the mean heart rate moves away from the resting heart rate of the sleep session. A higher value of α results in the aggregated inference being dependent on very few segments whose mean heart rate is closer to the resting heart rate of the sleep session. In one implementation, 0≤α≤0.5 and more particularly α=0.1 or α=0.2.

The one or more weighting rules may also, or instead, include a second weighting rule that weights an indicator of blood pressure value based on the temporal position (within the sleep session) of the segment of pulse data from which the indicator of blood pressure value was calculated. The second weighting rule assigns greater weights to indicator of blood pressure values calculated from segments of pulse data occurring towards the end of the sleep session than to those occurring towards the start of the sleep session. As such, the second weighting rule may assign a greater weight to the first indicator of baseline blood pressure value than to the second indicator of baseline blood pressure value when the first portion of the sleep session is temporally closer than the second portion of the sleep session to an end of the sleep session. For example, if the end of the sleep session occurs at time point t_{end} then the weight wⱼ assigned to a baseline blood pressure indicator value calculated from a segment of pulse data j occurring at time point tⱼ may be wⱼ=(t_{end}−tⱼ). The weight may be normalized by dividing the weight by (t_{end}−tₛₜₐᵣₜ) where tₛₜₐᵣₜ is the time point at the start of the sleep session. As with the first weighting rule, the second weighting rule may apply a weighting function (e.g., exponential function(s); sigmoid function(s); linear function(s), etc.) that may be parameterized by one or more parameters. For example,

$${w}_{j} = \frac{exp ( - α \cdot ( {t}_{e n d} - {t}_{j} ) )}{{Σ}_{j = 1}^{K} {w}_{j}}$$

where α is a parameter as described above in relation to the first weighting rule.

The one or more weighting rules may also, or instead, include a third weighting rule that weights an indicator of blood pressure value based on user's sleep stage at the time associated with the segment of pulse data from which the indicator of blood pressure value was calculated. That is, the third weighting rule may assign a greater weight to the first indicator of baseline blood pressure value than to the second indicator of baseline blood pressure value when the user is in a deeper sleep during the first portion of the sleep session than during the second portion of the sleep session. For example, a sleep stage classifier may be used to determine which stage (1-3) of sleep the user is in at a given time point. The weight applied by the third weighting rule may then correspond to the determined sleep stage such that a greater weight is applied when the user is in a deeper sleep (stage 3) than when they are in a lighter sleep (stage 1). As with the above weighting rules, the third weighting rule may apply a weighting function (e.g., exponential function(s); sigmoid function(s); linear function(s), etc.) that may be parameterized by one or more parameters (e.g., α).

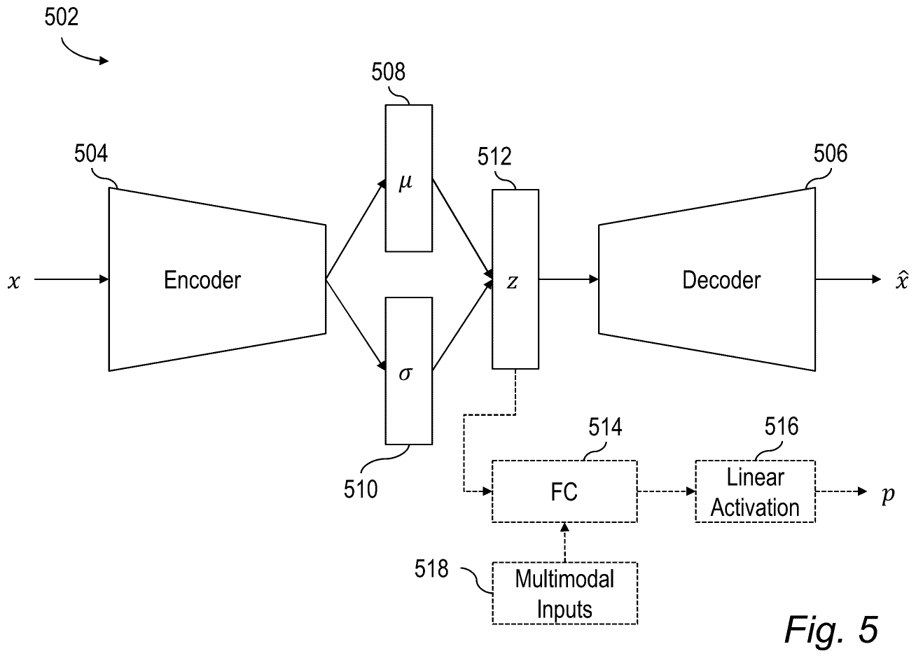

The one or more weighting rules may also, or instead, include a fourth weighting rule based on a quality score associated with quality of the pulse data used to generate an indicator of blood pressure value. That is, the fourth weighting rule may assign a greater weight to the first indicator of baseline blood pressure value than to the second indicator of baseline blood pressure value when the first segment of pulse data has a pulse quality value greater than the pulse quality value of the second segment of pulse data. The pulse quality value may be calculated using an encoder-decoder neural network (as described in more detail in relation to FIG. 5 below) and may be transformed by a weighting function (e.g., exponential function(s); sigmoid function(s); linear function(s), etc.) that may be parameterized by one or more parameters (e.g., α), as described above.

FIG. 2A is a flow chart illustrating a method 200 for baseline blood pressure estimation of a user of a wearable physiological monitor. The method 200 may be used in cooperation with any of the devices, systems, and methods described herein, such as by a user device (e.g., a mobile device) that is communicatively coupled to a wearable, continuous physiological monitoring device. The user device may include any of the user devices 1220 that are communicatively coupled to the physiological monitor 1206, as illustrated in FIG. 12. In general, the method 200 predicts an indicator of baseline blood pressure for the user using static and dynamic features extracted from the user's pulse data obtained while the user is at rest. As such, the method 200 provides an efficient and non-invasive approach for predicting an indicator of baseline blood pressure for a user—either a baseline blood pressure value (e.g., a baseline systolic or diastolic blood pressure value) or a hypertensive classification score (e.g., a systolic or diastolic hypertension classification)—from pulse data obtained from the user while the user is at rest.

In one embodiment, the method 100 shown in FIG. 1A is performed as part of, or combined with, the method 200 shown in FIG. 2A. That is, step 102 of the method 100 may correspond to step 202 of the method 200, step 104 of the method 100 may be performed as part of step 204 of the method 200, step 106 of the method 100 may correspond to step 212 of the method 200, and step 108 of the method 100 may correspond to step 214 of the method 200. As such, the skilled person will appreciate that steps of the method 200 may be combined with those of the method 100 and vice versa.

As shown in step 202, the method 200 may include identifying a segment of pulse data related to cardiac activity of a user during a portion of a sleep session. In one embodiment, step 202 corresponds to step 102 of the method 100 as described above. The segment of pulse data is obtained by a wearable physiological monitor. For example, the segment of pulse data may be obtained from a physiological monitor such as the physiological monitor 1206 in FIG. 12 by a user device such as the one or more user devices 1220. More particularly, the sequence of pulse data may be obtained from a suitable sensor (e.g., a photoplethysmography (PPG) sensor or electrocardiography (ECG) sensor) coupled to the physiological monitor. Alternatively, the segment of pulse data is obtained from a storage location such as a memory location or persistent storage location.

The segment of pulse data comprises a sequence of pulses (e.g., heart pulse samples or heart beats) that relate to the cardiac activity of the user while the user sleeps. That is, the segment of pulse data corresponds to a waveform or time series of values characterizing the cardiac activity of the user during a portion of a sleep session of the user. The portion of the sleep session corresponds to a subframe or time period of the sleep session (e.g., a 20 second portion, 30 second portion, 40 second portion, etc.). As such, the number of pulses within the segment of pulse data depends on the length of the portion of the sleep session. In one embodiment, the portion of the sleep session is chosen such that the segment of pulse data comprises in the range of 20 to 40 pulses.

In one embodiment, prior to step 204 the segment of pulse data is pre-processed or otherwise transformed. The method 200 may include normalizing each pulse within the segment of pulse data. Each pulse may be normalized with respect to pulse amplitude and/or with respect to pulse length. For example, each pulse within the segment of pulse data may be normalized such that the pulse within the segment of pulse data having the highest amplitude is normalized to have an amplitude of 1 and the pulse within the segment of pulse data having the longest pulse length is normalized to have a pulse length of 1, that occurs in a known manner. Additionally, or alternatively, the method 200 may include filtering, or selecting, pulses within the segment of pulse data such that pulses that satisfy one or more criteria are maintained for further analysis (e.g., are provided to step 204). The criteria may include a quality criterion that is satisfied when a pulse is determined to have one or more pulse characteristics such as a dicrotic notch. For example, a dicrotic notch classifier (e.g., as described in relation to FIG. 5 below) may be used to assign a dicrotic notch extent score to each pulse within a segment of pulse data and pulses having a dicrotic notch extent score above a threshold value (e.g., a score that indicates that the pulse has a dicrotic notch with a probability of 0.5, 0.75, 0.9, 0.95, or the like) are maintained for further analysis. In one embodiment, one or more further segments of pulse data are obtained until the number of pulses maintained for further analysis is above a threshold number (e.g., 40 pulses, 50 pulses, etc.).

As shown in step 204, the method 200 may include extracting, from the segment of pulse data, one or more static features that characterize an average pulse morphology during the portion of the sleep session. In one embodiment, step 104 of the method 100 is performed as part of step 204.

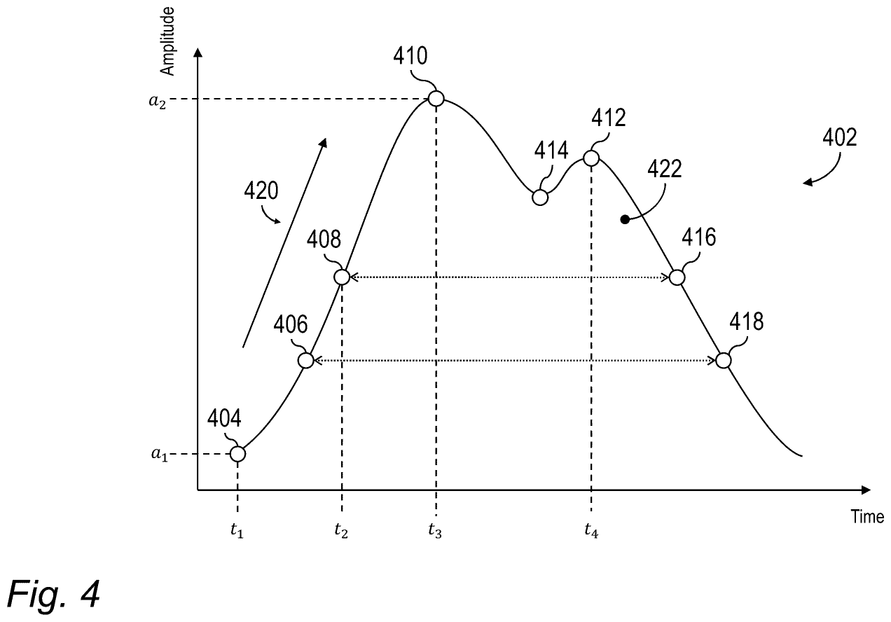

The one or more static features include one or more of: an average pulse width value; an average maximum acceleration value; an average maximum derivative value; an average time to maximum derivative or acceleration value; an average area under the curve value; an average area without detrending value; and an average time between systolic and diastolic peaks value. These features are described in more detail in relation to FIG. 4 below.

As shown in step 206, the method 200 may include extracting, from the segment of pulse data, one or more dynamic features that characterize temporal variation in pulse morphology during the portion of the sleep session. The one or more dynamic features are generated from a plurality of morphology features extracted from each pulse in the segment of pulse data.

The plurality of morphology features extracted for a pulse of the segment of pulse data include: a pulse width value; a maximum acceleration value; a maximum derivative value; a time to maximum value; a time to maximum acceleration value; an area under the curve value; an area without detrending value; a time between systolic and diastolic peaks value; an instantaneous pulse rate value; a pulse amplitude value; one or more latent features; and a notch metric value indicative of an extent of a dicrotic notch. In one embodiment, the one or more latent features are determined using an encoder-decoder neural network and the notch metric value is extracted using an encoder-decoder neural network. As described in more detail below in relation to FIG. 5, the encoder-decoder neural network may be a variational autoencoder and may be trained on a dataset of synthetic pulses.

As shown in step 208, in one embodiment, the dynamic features are generated from the plurality of morphology features by providing the plurality of morphology features to a Neural Network (NN) to generate the one or more dynamic features. The NN is trained to output dynamic features from pulse morphology features provided as input. The NN is a Recurrent Neural Network (RNN), such as a stacked Long Short Term Memory (LSTM) network or a Gated Recurrent Unit (GRU) network, a Convolutional Neural Network (CNN), or a Transformer based network. Further details regarding such networks are provided in relation to FIG. 3B below.

As shown in step 210, the method 200 may include identifying sleep data that characterizes the portion of the sleep session in relation to the sleep session. In one embodiment, step 210 is performed as the first step of the method 200 (i.e., step 210 is performed before step 202). The sleep data comprises sleep onset data indicative of a start time of the portion of the sleep session in relation to a start of the sleep session. Additionally, or alternatively, the sleep data comprises sleep stage data.

As shown in step 212, the method 200 may include identifying a machine learning model trained to receive as input one or more features obtained during a first time period and predict an indicator of blood pressure during a second time period. In one embodiment, the one or more features include a resting heart rate of the user (as described in relation to step 106 of the method 100 of FIG. 1A).

As described in more detail below in relation to FIGS. 3A and 3B, any suitable machine learning model may be used to predict an indicator of blood pressure from the one or more features. For example, a neural network comprising a concatenation layer, at least one dense layer, and one or more output layers (including an output layer for predicting a baseline blood pressure value and/or an output layer for predicting a hypertensive classification score). The machine learning model is trained on a training data set of features with corresponding baseline blood pressure indicator values. That is, the training data set comprises a set of training instances, where each training instance comprises one or more features and a corresponding baseline blood pressure indicator value. The one or more features are obtained during a first time period (e.g., at nighttime and/or while the user is asleep) and the corresponding baseline blood pressure indicator value is obtained during a second time period (e.g., at daytime and/or while the user is awake). Training a machine learning model on such a data set enables the (trained) machine learning model to predict an indicator of blood pressure value for one time point from features obtained at a second, earlier, time point. The training data set may comprise further feature values (e.g., pulse morphology features) and may also include multiple baseline blood pressure indicator values (e.g., systolic blood pressure, diastolic blood pressure, systolic hypertension indicator value, etc.).

The method 200 may include, prior to step 214, the step of training the machine learning model on a training data set (not shown). The skilled person will appreciate that the machine learning model can be trained in any suitable manner using a training approach suitable for the machine learning model or algorithm used. Further details regarding training of an artificial neural network are provided in relation to FIGS. 3A and 3B below.

As shown in step 214, the method 200 may include providing one or more features to the machine learning model to obtain an indicator of baseline blood pressure for the user. The one or more features provided as input to the machine learning model include the resting heart rate value extracted from the segment of pulse data (as described in relation to step 108 of the method 100 of FIG. 1A). Additionally, or alternatively, the one or more features provided as input to the machine learning model include the one or more static features extracted at step 204. Additionally, or alternatively, the one or more features provided as input to the machine learning model include the one or more dynamic features extracted at step 206. Additionally, or alternatively, the one or more features provided as input to the machine learning model include features that characterize demographic data of the user (e.g., height, age, weight, ethnicity, gender, skin tone, body mass index, etc.). In general, demographic data for a user may be reported by the user, e.g., as manual input, or demographic data for the user may be inferred based on other data from the user, or data from other sources. Additionally, or alternatively, the one or more features provided as input to the machine learning model include the sleep data identified at step 210. Additionally, or alternatively, the one or more features provided as input to the machine learning model include pulse arrival time features. In one embodiment, the pulse arrival time features are collected while the user is at rest. Additionally, or alternatively, the one or more features provided as input to the machine learning model include pulse arrival time variation features. In one embodiment, the pulse arrival time variation features are collected during deep respirations of the user.

The indicator of baseline blood pressure obtained from the machine learning model is an indicator of a blood pressure value that would be obtained for the user if measured under standard conditions. The indicator of baseline blood pressure may be calculated and/or reported in a variety of forms, e.g., as a baseline mean blood pressure, a baseline diastolic blood pressure, a baseline systolic blood pressure, a baseline diastolic-to-systolic range, or some combination of these. In this context, the mean blood pressure is the average arterial pressure throughout the cardiac cycle (and in an example, may be estimated by diastolic pressure+⅓ (systolic pressure-diastolic pressure), or diastolic pressure+⅓ (pulse pressure)), however, other measures of mean blood pressure may also or instead be used. Alternatively, the indicator of baseline blood pressure may be expressed as an average of the baseline systolic blood pressure value, the baseline diastolic blood pressure value, and/or the baseline mean blood pressure.

As a significant advantage, this indicator of baseline blood pressure may be based on physiological data such as cardiac data acquired in the background from a user while at rest (e.g., while sleeping), and the machine learning model may be configured to provide a value that accurately indicates what the user's blood pressure would be if measured under standard conditions while the user is awake during the day. As a further advantage, obtaining the indicator of baseline blood pressure based on nighttime cardiac activity with the techniques described herein foregoes the need for careful management of testing conditions for daily testing or the like.

It will be understood that the suggested conditions for measuring blood pressure, as outlined by various health organizations such as the American Heart Association (AHA) and the European Society of Hypertension (ESH), do not have a specific, singular name that encompasses the entire set of guidelines. However, the procedures outlined by these organizations do have a number of characteristics in common. In the context of this disclosure, the standard conditions are any such conditions useful for an accurate clinical blood pressure measurement, typically including, e.g., proper body positioning (e.g., sitting with feet flat on the floor), proper arm positioning (supported on a flat surface, with the upper arm at heart level), proper timing (e.g., after sitting quietly after at least five minutes before measurement), proper preparation (no stimulants or exercise for at least 30 minutes before measurement), and where appropriate, the use of a properly calibrated monitor (where automated measurements are taken) and properly sized cuff. While it is possible to take blood pressure measurements and obtain accurate results under other various conditions, these constraints help to ensure that the result is consistent, and provide an accurate benchmark for assessing hypertension and other conditions related to blood pressure. In general, any such procedure(s) are referred to herein as “a standardized blood pressure measurement technique” or “a standard protocol for blood pressure measurement.”

The method 200 may include the step of tracking one or more trends in baseline blood pressure for the user based on the baseline blood pressure value and one or more historical blood pressure values of the user. The one or more trends may be tracked over a time period (e.g., daily, weekly, monthly, etc.). As such, the one or more historical blood pressure values of the user may be baseline blood pressure values of the user obtained over a preceding time period (e.g. one day prior to determining the baseline blood pressure value, or 7 days prior to determining the baseline blood pressure value, or one month prior to determining the baseline blood pressure, etc.) such that the one or more trends may be tracked over the preceding time period. Tracking the one or more trends may include displaying the one or more historical baseline blood pressure values and the current baseline blood pressure value for presentation to a user and/or identifying trends in the one or more historical baseline blood pressure values and the baseline blood pressure value (e.g., identifying periodicity or repeating patterns, identifying increases or decreases, etc.). In one embodiment, the indicator of baseline blood pressure is calculated from an average of daily baseline blood pressure measurements over a number of days (e.g., a trailing 7 day average). An indicator of baseline blood pressure is obtained from the machine learning model each day (as described above) and the average over the number of days is calculated and reported as the overall indicator of baseline blood pressure.

Additionally, or alternatively, the indicator of baseline blood pressure is a hypertensive classification score for the user. The hypertensive classification score is one of a systolic hypertension classification score or a diastolic hypertension classification score.

The method 200 may include the step of displaying, on a device of the user, an alert associated with the indicator of baseline blood pressure. For example, the alert may take the form of a blood pressure reading (e.g., either or both of the estimated systolic and diastolic blood pressure) that is presented to a user within an application running on a user device such as the one or more devices 1220 shown in FIG. 12. The blood pressure estimation may be displayed alongside a history of estimated blood pressure readings thereby allowing the user to monitor and track changes in blood pressure over time. This can provide important health insights to the user and may allow them to relate specific factors to changes in blood pressure over time. Additionally, or alternatively, the alert may take the form of a notification, message, or warning presented on a user device such as the one or more devices 1220 shown in FIG. 12. The alert may be shown when the indicator of baseline blood pressure satisfies a predetermined alert criterion. For example, the predetermined alert criterion may be a threshold blood pressure value such that if the user's estimated blood pressure is elevated above this value, then the alert is displayed. As a further example, the predetermined alert criterion may be a specific classification score or value such that if the user's estimate hypertension classification score is above, or the same as, the predetermined alert criterion, then the alert is displayed.

FIG. 2B is a flowchart illustrating a method 216 for aggregated baseline blood pressure estimation. The method 216 may be used in cooperation with any of the devices, systems, and methods described herein, such as by a user device (e.g., a mobile device) that is communicatively coupled to a wearable, continuous physiological monitoring device. The user device may include any of the user devices 1220 that are communicatively coupled to the physiological monitor 1206, as illustrated in FIG. 12. In general, the method 216 determines an aggregated indicator of baseline blood pressure from multiple segments of pulse data obtained over a period of time (e.g., a sleep session). As such, the method 216 provides an efficient and non-invasive approach for predicting an indicator of baseline blood pressure for a user—either a baseline blood pressure value (e.g., a baseline systolic or diastolic blood pressure value) or a hypertensive classification score (e.g., a systolic or diastolic hypertension classification)—from pulse data obtained from the user while the user is at rest. Aggregating baseline indicators of blood pressures from across a period of time (e.g., a sleep session) may help obtain a more robust and accurate estimate of the user's blood pressure and physiological state.

As shown in step 218, the method 216 may include identifying multiple segments of pulse data of a user during a sleep session. For example, 100 segments of pulse data, 200 segments of pulse data, or 300 segments of pulse data may be identified from a larger sequence of pulse data recorded for the user during the sleep session. As described in more detail above, the segments of pulse data may comprise 20-40 pulses and may be obtained by a wearable physiological monitor (e.g., the physiological monitor 1206 shown in FIG. 12).

As shown in step 220, the method 216 may include obtaining multiple estimated baseline indicators of blood pressure for the user. That is, the multiple segments of pulse data identified at step 218 may be used to determine an associated indicator of baseline blood pressure. For example, the method 200 shown in FIG. 2A may be employed for each of the multiple segments of pulse data to determine multiple associated baseline indicators of blood pressure. Therefore, the skilled person will appreciate that step 220 may include performing the method 200 shown in FIG. 2A for multiple segments of pulse data.

As shown in step 222, the method 216 may include aggregating the multiple estimated baseline indicators of blood pressure. For example, the aggregated indicator of baseline blood pressure may be the average indicator of baseline blood pressure calculated from the multiple baseline indicators of blood pressure obtained at step 220. Alternatively, the aggregated indicator of baseline blood pressure may be obtained using a weighted aggregation strategy such as that described in relation to step 112 of the method 100 above. Therefore, the step 222 may correspond in operation to the step 112 of the method 100 such that the above description of step 112 also applies to the operations performed at step 222.

The aggregated indicator of baseline blood pressure may then be output, displayed, stored, or otherwise processed as described above in relation to the method 200 shown in FIG. 2A.

FIG. 3A illustrates a portion of a system for baseline blood pressure estimation of a user of a wearable physiological monitor. FIG. 3A shows wearable physiological monitor 302, pulse data 304, a feature extraction module 306, one or more static features 308 that include a resting heart rate value 310, one or more dynamic features 312, and one or more additional features 314. FIG. 3A further shows one or more machine learning models 316, a first indicator of baseline blood pressure 318, and a second indicator of baseline blood pressure 320. In one embodiment the portion of the system is used to carry out the methods described in relation to FIGS. 1 and 2 above.

The wearable physiological monitor 302 is any suitable physiological monitor that can obtain pulse data from a user (e.g., a user wearing the physiological monitor). For example, the wearable physiological monitor 302 may be the physiological monitor 1206 in FIG. 12. The pulse data 304 obtained from the wearable physiological monitor 302 represents the cardiac activity of the user. For example, the pulse data 304 may be a segment of pulse data related to cardiac activity of the user during a time period such as a portion of a sleep session. The pulse data 304 comprises a waveform or time-series of values that characterize the cardiac activity of the user and so comprises one or more pulses appearing as peaks within the pulse data 304 (as illustrated in FIG. 4). In one embodiment, the pulse data 304 comprises between 20 and 40 pulses (i.e., heart beats).

The pulse data 304 is provided to the feature extraction module 306 to extract one or more features that characterize the pulse data, and which can be provided to the one or more machine learning models 316 to determine an indicator of baseline blood pressure for the user. The one or more features extracted by the feature extraction module 306 include a resting heart rate value 310. Additionally, or alternatively, the one or more features include one or more static features 308 and/or one or more dynamic features C12.

In one embodiment, the pulse data 304 is pre-processed prior to being analyzed by the feature extraction module 306. The pre-processing may include applying one or more filters to the pulse data 304 (e.g., a low-pass filter, a high-pass filter, a moving average filter, etc.), normalizing the pulse data 304, and/or selecting pulses within the pulse data 304. For example, each pulse may be normalized with respect to pulse amplitude and/or with respect to pulse length. As a further example, pulses within the segment of pulse data may be selected such that pulses that satisfy one or more criteria are maintained for further analysis (e.g., pulses that are determined to have a distinct dicrotic notch).

The feature extraction module 306 extracts the resting heart rate value 310 from the pulse data 304 using a pulse or beat counting algorithm to calculate the number of pulses within the segment of pulse data. The number of pulses within the pulse data 304 is then divided by the length (in minutes) of the pulse data 304 to calculate the resting heart rate value 310 (in beats per minute).

The one or more static features 308 extracted from the pulse data 304 by the feature extraction module 306 characterize an average pulse morphology over the time period of the pulse data 304 (e.g., during the portion of the sleep session). That is, the feature extraction module 306 identifies one or more pulses within the pulse data 304 and calculates one or more features (morphologic features) for the one or more pulses. Average values for the one or more features calculated across the one or more pulses are provided as the one or more static features 308. The one or more static features 308 extracted by the feature extraction module 306 include one or more of an average pulse width value; an average maximum acceleration value; an average maximum derivative value; an average time to maximum derivative or acceleration value; an average area under the curve value; an average area without detrending value; and an average time between systolic and diastolic peaks value. These features are described in more detail in relation to FIGS. 4 and 5 below. In one embodiment, the one or more static features 308 include one or more latent features determined using an encoder-decoder neural network (as described below in relation to FIG. 5). In one implementation, the one or more static features 308 are pulse width at 75%, median heart rate, time to maximum derivative, time to maximum acceleration, respiratory rate, time between systolic and diastolic peaks, and two latent features.

The one or more dynamic features 312 extracted from the pulse data 304 by the feature extraction module 306 characterize temporal variation in pulse morphology over the time period of the pulse data 304 (e.g., during the portion of the sleep session). That is, the one or more dynamic features 312 capture sequential information—i.e., the change or transition from one pulse to another within the pulse data 304. The one or more dynamic features 312 are calculated from a plurality of morphology features extracted from pulses within the pulse data 304. The morphology features are provided to a trained Recurrent Neural Network (RNN) to generate the one or more dynamic features 312 that characterize the temporal variation or change in the pulse morphology features over the pulse data 304 (as described in more detail in relation to FIG. 3B below). The plurality of morphology features extracted for a pulse of the pulse data 304 include: a pulse width value; a maximum acceleration value; a maximum derivative value; a time to maximum value; a time to maximum acceleration value; an area under the curve value; an area without detrending value; a time between systolic and diastolic peaks value; an instantaneous pulse rate value; a pulse amplitude value; one or more latent features; and/or a notch metric value indicative of an extent of a dicrotic notch. These features are described in more detail in relation to FIGS. 4 and 5 below.

In one embodiment, one or more additional features 314 are obtained and provided as input to the one or more machine learning models 316. The one or more additional features 314 include sleep data, demographic data, and/or contextual data.

When the pulse data 304 corresponds to pulse data obtained from a user during a portion of a sleep session of the user, the sleep data included within the one or more additional features 314 characterizes the portion of the sleep session in relation to the sleep session. For example, the sleep data may comprise sleep onset data indicative of a start time of the portion of the sleep session in relation to a start of the sleep session. That is, if the sleep session is identified as starting at 22:00 and the portion of the sleep session is identified as starting at 02:00, then the start time would be calculated as the difference in hours between the two times (i.e., 4 hours). As a further example, the sleep data may comprise sleep stage data that indicates the stage of sleep that the user was in when the pulse data 304 was obtained (e.g., REM, Light Sleep, Deep Sleep, or REM, Stage 1, Stage 2, etc.).

The demographic data characterizes demographic data or information of the user (e.g., weight, height, body mass index, etc.). In one embodiment, the demographic data is obtained from one or more data sources associated with, or within, the physiological monitoring system such as a user profile, exercise diary, nutrition diary, and the like.

The contextual data characterizes the context within which the pulse data 304 was obtained. That is, the contextual data encodes additional factors that may affect the user's baseline blood pressure that are not represented within the static, dynamic, or additional features. These factors generally relate to the activity of the user within a time period prior to the pulse data 304 being obtained (e.g., 4 hours, 8 hours, 12 hours, 24 hours, etc.) and/or to the user's medical history. In one embodiment, the contextual data comprises a set of flags or indicator values related to contextual conditions. For example, for the contextual condition “the user has a history of hypertension”, the corresponding flag would be set to 1 if the user satisfies that contextual condition and the flag would be set to 0 if not. Examples of contextual conditions include whether the user consumed above a threshold amount of stimulants, such as caffeine or tobacco, within a time period prior to the pulse data 304 being obtained, whether the user exercised within a time period prior to the pulse data 304 being obtained, whether the user has indicated an expected change in sleep patterns (e.g., due to travel or entertainment activities), whether the user has placed the wearable physiological monitor on a non-standard position when obtaining the pulse data 304 (e.g., on the bicep or ankle), whether the user has a history of high blood pressure (hypertension), whether the user has a history of coronary artery disease, whether the user has a history of obstructive sleep apnea, whether the user has a history of vascular disease such as atherosclerosis, whether the user has a history of any kidney problems, whether the user is taking medication to treat high blood pressure, etc.

The one or more features (e.g., one or more static features, one or more dynamic features, and/or one or more additional features) are provided to the one or more machine learning models 316 that are trained to receive one or more features as input and provide an indicator of blood pressure as output. More particularly, the one or more machine learning models 316 are trained to receive one or more features obtained during a first time period and predict an indicator of blood pressure during a second time period. For example, the one or more features may include a resting heart rate value obtained at nighttime (e.g., while the user is asleep) and the indicator of blood pressure may be a baseline systolic blood pressure value at daytime (e.g., while the user is awake).

The one or more machine learning models 316 include a trained machine learning model such as a trained regression model (for predicting baseline blood pressure values) or a trained classification model (for predicting a hypertensive classification score). In one embodiment, the one or more machine learning models 316 include a neural network as described in relation to FIG. 3B below. The neural network comprises an input layer, a hidden layer, and at least one output layer (one output layer for predicting baseline blood pressure values and/or one output layer for predicting a hypertensive classification score). The one or more machine learning models 316 are trained on a training data set of features with corresponding baseline blood pressure indicator values. That is, the training data set comprises a set of training instances, where each training instance comprises one or more features (e.g., one or more static features, one or more dynamic features, and/or one or more additional features) and one or more corresponding baseline blood pressure indicator values (e.g., a baseline blood pressure value and/or a hypertensive classification score). In one implementation, the training data set comprises approximately 130,000 segments of pulse data (each of approximately 30 seconds in length) for around 500 unique subjects along with corresponding blood pressure values (in bpm) and hypertensive classifications for each segment of pulse data. Static features and dynamic features were extracted from each segment of pulse data to construct a training data set of static and dynamic features upon which the one or more machine learning models were trained.

The one or more machine learning models 316 generate a first indicator of baseline blood pressure 318 and/or a second indicator of baseline blood pressure 320. For example, the first indicator of baseline blood pressure 318 may be a baseline systolic blood pressure value and the second indicator of baseline blood pressure 320 may be a baseline diastolic blood pressure value. Alternatively, the first indicator of baseline blood pressure 318 may be an indicator of baseline blood pressure value and the second indicator of baseline blood pressure 320 may be a hypertensive classification score.

The first indicator of baseline blood pressure 318 and/or the second indicator of baseline blood pressure 320 may then be provided to one or more components of the physiological monitoring system. For example, an indicator of baseline blood pressure indicative of an estimated blood pressure of the user may be output to a monitoring application of the physiological monitoring system (e.g., an application executing on a device such as the one or more devices 1220 of FIG. 12) that may then display an alert to a user if the estimated blood pressure meets a threshold condition (e.g., is elevated above a predetermined threshold). As a further example, an indicator of baseline blood pressure may be provided to a device (e.g., the one or more devices 1220 of FIG. 12) for storage. Stored blood pressure indicators may then be analyzed to track changes in the user's blood pressure over time.

FIG. 3B illustrates a portion of a neural network based system for baseline blood pressure estimation of a user of a wearable physiological monitor. The portion of the neural network based system comprises a first neural network 322 comprising a concatenation layer 324, a shared dense layer 326, a regression output layer 328, and a classification output layer 330. The portion of the neural network based system further comprises a second neural network 332, one or more non-dynamic features 334, a plurality of morphology features 336, a baseline blood pressure value 338, and a hypertensive classification score 340.

The skilled person will appreciate that the portion of the neural network based system shown in FIG. 3B corresponds to an embodiment of a portion of the system shown in FIG. 3A. That is, in one embodiment the first neural network 322 corresponds to the one or more machine learning models 316 of FIG. 3A. Additionally, or alternatively, the first neural network 322 corresponds to a dynamic feature extraction process of the feature extraction module 306. The one or more non-dynamic features 334 correspond to the one or more static features 308 and/or the one or more additional features 314 shown in FIG. 3A. The morphology features 336 correspond to the plurality of morphology features extracted from pulses within the pulse data 304 as described above in relation to FIG. 3A.

The first neural network 322 is trained to receive one or more features (e.g., the one or more non-dynamic features 334 and/or the one or more dynamic features generated by the second neural network 332) and predict the baseline blood pressure value 338 and/or the hypertensive classification score 340. The concatenation layer 324 of the first neural network 322 concatenates the one or more features. The outputs of the concatenation layer 324 are connected to the shared dense layer 326. The outputs of the shared dense layer 326 are connected to the regression output layer 328 and/or the classification output layer 330. In one implementation, the first neural network 322 has an architecture comprising an input layer of 9 nodes connected to the non-dynamic features 334 and the concatenation layer 324 has 19 nodes connected to the 9 nodes of the input layer and the 10 nodes of the second neural network 332 (as described below). The shared dense layer 326 comprises a single node connected to the 19 nodes of the concatenation layer 324 (and thus 20 parameters). The regression output layer 328 utilizes a linear activation function and the classification output layer 330 utilizes a sigmoid activation function.

The second neural network 332 is configured to extract one or more dynamic features from the plurality of morphology features 336. The second neural network 332 is used in conjunction with the first neural network 322. Alternatively, the second neural network 332 is used to provide dynamic features to one or more other machine learning models (e.g., a linear regression model, a support vector machine, etc.). The second neural network 332 is operable to characterize temporal variation in pulse morphology from the morphology features 336.

In one embodiment, the second neural network 332 is a stacked Long Short Term Memory (LSTM) network. The LSTM network comprises three LSTM layers coupled in sequence. The number of nodes within the final LSTM layer of the LSTM network determines the number of dynamic features provided as output. The number of nodes is chosen such that between 5 and 20 dynamic features are provided as output and, in one implementation, 10 nodes are provided so as to provide 10 dynamic features as output. The architecture of the second neural network 332 when implemented as a stacked LSTM network according to one embodiment is shown in Table I. The output of the final LSTM layer is connected to the concatenation layer 324 of the first neural network 322 (as described above).

TABLE I

| Layer | Shape | Parameters |
|---|---|---|
| Input | (40, 14) | 0 |
| Masking | (40, 14) | 0 |
| LSTM | (40, 10) | 1000 |
| LSTM | (40, 10) | 840 |
| LSTM | 10 | 840 |

In one embodiment, the second neural network 332 is a Gated Recurrent Unit (GRU) network. The GRU network has a similar architecture to the stacked LSTM network described above but with the stacked LSTM layers being replaced with stacked GRU layers. The architecture of the second neural network 332 when implemented as a stacked GRU network according to one embodiment is shown in Table II. The output of the final GRU layer is connected to the concatenation layer 324 of the first neural network 322 (as described above).

TABLE II

| Layer | Shape | Parameters |
|---|---|---|
| Input | (40, 14) | 0 |
| Masking | (40, 14) | 0 |
| GRU | (40, 10) | 780 |
| GRU | (40, 10) | 660 |
| GRU | 10 | 660 |

The first neural network 322 and the second neural network 332 are trained on a training data set as described above in relation to the one or more machine learning models 316 in FIG. 3A. In one implementation, the first neural network 322 is trained using an Adam optimizer with a learning rate of 0.0005, exponential decay rates for the first and second moment estimates of 0.9 and 0.999 respectively, and ε=1×10⁻⁸. In one implementation, pre-training is performed such that the weights of the first neural network 322 are frozen and only the weights of the second neural network 332 are trained. The weights of the first neural network 322 are then un-frozen and the weights of both the first neural network 322 and the second neural network 332 are trained (with the pre-trained weight values of the second neural network 332 used as initial values). A mean squared error loss function is used to evaluate the outputs of the regression output layer 328 and a binary cross entropy loss function is used to evaluate the outputs of the classification output layer 330.

FIG. 3C shows a portion of a system for aggregated baseline blood pressure estimation. The portion of the system shown in FIG. 3C may be used to carry out the steps of either of the methods shown in FIGS. 1B and 2B. FIG. 3C shows segments of pulse data 342, a plurality of estimators 344-1, 344-2, 344-3, an aggregator 346, and an aggregated indicator of baseline blood pressure value 348. FIG. 3C further shows a first segment of pulse data 350, a first weight 352, a second weight 354, a first indicator of baseline blood pressure value 356, a first function 358, a second function 360, and a first output indicator of baseline blood pressure value 362.

The segments of pulse data 342 may include multiple segments of pulse data obtained during a sleep session of the user. For example, the segments of pulse data 342 may include 100 segments of pulse data, 200 segments of pulse data, or 300 segments of pulse data identified from a larger sequence of pulse data recorded for the user during the sleep session. Each segment of pulse data may comprise between 20 and 40 pulses (heartbeats). As described in more detail above, the segments of pulse data may be obtained by a wearable physiological monitor (e.g., the physiological monitor 1206 shown in FIG. 12). The segments of pulse data 342 include the first segment of pulse data 350 that is passed to the estimator 344-1 to determine an indicator of baseline blood pressure value. Further segments of pulse data are passed to estimators 344-2, 344-3 to obtain further indicator of baseline blood pressure values. That is, each of the segments of pulse data 342 are passed to an estimator to calculate a corresponding indicator of baseline blood pressure value. These values are aggregated by the aggregator 346 to determine the aggregated indicator of baseline blood pressure value 348.

While FIG. 3C illustrates that each of the plurality of estimators 344-1, 344-2, 344-3 are separate/independent modules or units, they may in some examples be the same estimator but applied sequentially or in parallel to the different segments of pulse data (e.g., using a threaded architecture or the like). As shown for the estimator 344-1, an estimator generates an indicator of baseline blood pressure value (e.g., the estimator 344-1 generates the first indicator of baseline blood pressure value 356) from a segment of pulse data (e.g., the first segment of pulse data 350). These values may be generated using the system described in relation to FIGS. 3A and 3B above. As such, the estimators shown in FIG. 3C may correspond to blood pressure estimation system shown in FIGS. 3A and 3B. A weighting strategy is used to determine a weight to be applied to the indicator of baseline blood pressure value to generate the output or weighted indicator of baseline blood pressure value for the estimator. In the example shown in FIG. 3C, a first weighting rule defined according to a first weight 352 and a first function 358 and a second weighting rule defined according to a second weight 354 and a second function 360 are used to determine the overall weight to be applied to the first indicator of baseline blood pressure value 356 to generate the first output indicator of baseline blood pressure value 362. For example, the first weight 352 may be a resting heart rate deviation (as described above in relation to FIG. 1B) and the first function 358 may be a sigmoid activation function applied to the first weight 352, and the second weight 354 may be a pulse quality weighting (as described above in relation to FIG. 1B) and the second function 360 may be a sigmoid activation function applied to the second weight 354. The parameters of the sigmoid activation functions may be manually set or learnt during a training phase (e.g., optimal values for the parameters are determined using a training data set of pulse data and associated blood pressure values). The outputs of the two functions may be summed or multiplied and may also be normalized before being multiplied with the first indicator of baseline blood pressure value 356. In one example, the normalization of weights is performed as part of the aggregation performed by the aggregator 346 such that the weights are normalized across the different estimators.

The aggregator 346 aggregates the outputs of the plurality of estimators 344-1, 344-2, 344-3 to determine the aggregated indicator of baseline blood pressure value 348. For example, the aggregator 346 may sum all the outputs from the plurality of estimators 344-1, 344-2, 344-3 or may normalize the weights, multiply the estimated baseline blood pressure indicator values obtained from the plurality of estimators 344-1, 344-2, 344-3 with the normalized weights, and then sum the weighted values.

Advantageously, the aggregation system shown in FIG. 3C efficiently determines an accurate and robust indicator of baseline blood pressure value (e.g., baseline systolic/diastolic blood pressure) that may be used to identify trends in blood pressure over time. Moreover, the rule-based weighting strategy may be extended or limited without requiring any adaptation of the underlying strategy thereby enabling efficient deployment, maintenance, and optimization of the system.

FIG. 4 illustrates feature points that can be extracted from a pulse of a segment of pulse data. FIG. 4 shows a pulse 402 that may form a part of a segment of pulse data (e.g., the segment of pulse data 304 in FIG. 3A). FIG. 4 further shows a first ascension point 404, a second ascension point 406, and a third ascension point 408. FIG. 4 also shows a systolic peak 410, a diastolic peak 412, and a dicrotic notch 414. FIG. 4 further shows, a first descension point 416, a second descension point 418, a maximum acceleration vector 420, and an area under the curve 422.

The first ascension point 404 corresponds to the point of the pulse 402 at time t₁ where the pulse begins to rise from a baseline level. The pulse 402 has amplitude a₁ at time t₁. The second ascension point 406 and the third ascension point 408 correspond to the points during the ascension of the pulse 402 (i.e., before the pulse 402 has reached maximum amplitude) at which the pulse 402 has reached approximately 25% and 50% of its maximum amplitude respectively. As shown in FIG. 4, the pulse 402 reaches 50% of its maximum amplitude at time t₂. The pulse 402 is generally composed of two components—a systolic component followed by a diastolic component. The systolic component arises from a forward-going pressure wave along the left ventricle while the diastolic component arises from pressure wave transmitted along the aorta (Millasseau, S. C., Kelly, R., Ritter, J., and Chowienczyk, P. (2002). Determination of age-related increases in large artery stiffness by digital pulse contour analysis. Clin. Sci. 103, 371-377). The systolic peak 410 of the systolic component corresponds to the point in the pulse 402 that represents the maximum blood volume during each cardiac cycle. Typically, the systolic peak corresponds to the point within a pulse that has the largest amplitude. In the example shown in FIG. 4, the systolic peak 410 occurs at time t₃ and has amplitude a₂. The diastolic peak 412 of the diastolic component corresponds to the highest amplitude during the diastolic component following the systolic peak 410. The dicrotic notch 414 corresponds to the inflection point between the systolic peak 410 and the diastolic peak 412. The first descension point 416 and the second descension point 418 correspond to the points during the descension of the pulse 402 (i.e., after the pulse 402 has reached maximum amplitude) at which the pulse 402 has reached approximately 50% and 25% of its maximum amplitude.

The pulse 402 may be extracted from a segment of pulse data by utilizing a peak finding algorithm on the inverted segment of pulse data (e.g., the inverted PPG signal). The peaks identified using the peak finding algorithm are then used to define the boundaries between each pulse within the segment of pulse data. An example peak finding algorithm is the local maxima algorithm based on a comparison of neighboring values. The systolic peak 410, the diastolic peak 412, and the dicrotic notch 414 may be identified from the pulse 402 by identifying key points within the second derivative of the pulse 402. For example, the time point associated with the minimum value of the second derivative may be used to identify the time point in the pulse 402 associated with the systolic peak 410, the time point associated with the first peak of the second derivative following the minimum value may be used to identify the time point associated with the dicrotic notch 414, and the time point associated with the minimum value of the second derivative after the dicrotic notch may be used to identify the time point associated with the diastolic peak 412. In one embodiment, the dicrotic notch 414 is identified using an encoder-decoder approach as described below in relation to FIG. 5.

The feature points shown in FIG. 4 are used to determine one or more morphology features that are used to determine static and/or dynamic features (as described above). As previously stated, morphology features include a pulse width value, a maximum acceleration value, a maximum derivative value, a time to maximum value, a time to maximum acceleration value, an area under the curve value, an area without detrending value, a time between systolic and diastolic peaks value, an instantaneous pulse rate value, a pulse amplitude value, one or more latent features, and a notch metric value indicative of an extent of a dicrotic notch.

Pulse width characterizes the width of the pulse 402 at a specific height (amplitude). The pulse width value may be calculated by measuring the difference (in time) between two points on the pulse 402 that have substantially the same amplitude. For example, the pulse width at 50% is calculated as the time difference between the third ascension point 408 and the first descension point 416 and the pulse width at 25% is determined as the time difference between the second ascension point 406 and the second descension point 418. Multiple pulse widths can be calculated to characterize the width of the pulse at different points of the pulse 402 (e.g., 10% pulse width, 25% pulse width, etc.). In one implementation, pulse width at 75% is used.

Maximum acceleration corresponds to the maximum rate of change of the pulse 402 during ascension (i.e., between time t₁ and t₂). In FIG. 4, the maximum acceleration of the pulse 402 is shown by the maximum acceleration vector 420. The maximum derivative corresponds to the largest derivative calculated from the pulse 402. That is, the derivative may be computed at all points across the pulse 402 and the largest value reported as the maximum derivative value for the pulse 402. The time to maximum value characterizes the time taken for the pulse 402 to reach maximum amplitude. That is, the time to maximum value corresponds to the time between the first ascension point 404 and the systolic peak 410 (i.e., t₃−t₁). The time to maximum acceleration characterizes the time taken for the pulse 402 to reach its maximum acceleration. That is, the time to maximum acceleration corresponds to the time between the first ascension point 404 and the time associated with the point at which the pulse 402 reaches maximum acceleration.

The area under the curve 422 is a measure of the area under the pulse 402 (as indicated by the shaded region under the pulse 402 in FIG. 4). The area without detrending value is a measure of the area under the pulse 402 without correction of baseline wander. Specifically, the area under the portion of the pulse 402 from the first ascension point 404 to the point at which the pulse 402 first starts detrending (i.e., the systolic peak 410).

The time between systolic and diastolic peaks measures the time taken for the pulse 402 to transition from the systolic peak 410 to the diastolic peak 412 (i.e., t₄−t₃). The instantaneous pulse rate value corresponds to the pulse rate value determined over a short time frame (e.g., using the time difference between two pulses, three pulses, four pulses, etc.). For example, the instantaneous pulse rate value calculated from two pulses occurring 0.5 seconds apart is calculated as 60/0.5=120 bpm. The pulse amplitude value corresponds to the highest amplitude of the pulse 402 (i.e., the amplitude of the systolic peak 410). The one or more latent features and the notch metric value correspond to features extracted from an encoder-decoder network, as described in more detail below in relation to FIG. 5.

FIG. 5 shows an encoder-decoder network 502. The encoder-decoder network is a variational autoencoder (VAE) comprising an encoder network 504 and a decoder network 506. The encoder network 504 maps an input vector x to a mean vector 508 μ and a standard deviation vector 510 σ. A latent vector 512 z is sampled from the distribution defined by the mean vector 508 μ and the standard deviation vector 510 σ. The latent vector 512 z is a low-dimensional representation of the input vector x. The decoder network 506 generates a reconstructed input vector \hat{x} from the latent vector 512 z. In one embodiment, the encoder-decoder network 502 is a supervised VAE further comprising a fully connected layer 514 and a linear activation function 516. The fully connected layer 514 receives the mean vector 508, the standard deviation vector 510, and may also receive multimodal inputs 518.

In general, the encoder-decoder network 502 maps a high-dimensional signal x to a low-dimensional latent space. The high-dimensional signal is a pulse of a segment of pulse data (e.g., the segment of pulse data 304 of FIG. 3A). For example, if a pulse waveform is composed of 128 samples, the high-dimensional signal input to the encoder-decoder network 502 would be x∈ [symbol] ¹²⁸. The latent vector 512 z is sampled from a low-dimensional latent space and provides a compact characterization of the pulse. More particularly, the low-dimensional latent space provides greater separability between pulses having different pulse characteristics; for example, different extents of dicrotic notch. Here, the extent of a dicrotic notch represents the difference in amplitude between the systolic peak and dicrotic notch, and the diastolic peak and dicrotic notch. The latent vector 512 z may thus be considered to encode one or more characteristics of the pulse represented by the input vector x. In one embodiment, the latent vector 512 of a pulse is used as a morphology feature and/or a static feature (i.e., the latent vector 512 corresponds to the one or more latent features).

In one implementation, the encoder network 504 maps from a high-dimensional input vector x∈ [symbol] ¹²⁸ to a low dimensional latent space [symbol] ⁴ such that z∈ [symbol] ⁴. The encoder network 504 comprises an input layer, four convolutional layers, a flatten layer, a dense layer, and three output layers (an output layer for the mean vector, an output layer for the standard deviation vector and an output layer for the sampled latent vector). As shown in FIG. 5, the output layer for the mean vector (i.e., the mean vector 508) and the output layer for the standard deviation (i.e., the standard deviation vector 510) are both connected to the previous dense layer of the encoder network 504. The sample latent vector (i.e., the latent vector 512) is connected to the output layer for the mean vector and the output layer for the standard deviation vector. The architecture of the encoder network 504 is shown in Table III.

TABLE III

| Layer | Shape | Parameters |
|---|---|---|
| Input | (128, 1) | 0 |
| Gaussian Noise | (128, 1) | 0 |
| Convolutional 1D | (64, 8) | 88 |
| Convolutional 1D | (32, 16) | 1296 |
| Convolutional 1D | (16, 16) | 2576 |
| Convolutional 1D | (8, 16) | 2576 |
| Flatten | 128 | 0 |
| Dense | 16 | 2064 |
| Output (μ) | 4 | 68 |
| Output (σ) | 4 | 68 |
| Output (z) | 4 | 0 |

In the same implementation, the decoder network 506 reconstructs the high-dimensional reconstructed input vector \hat{x}∈ [symbol] ¹²⁸ from the low-dimensional latent vector z∈ [symbol] ⁴. The decoder network 506 comprises an input layer, a dense layer, a reshape layer, four 1-dimensional convolutional transpose layers, and a dense layer. The architecture of the decoder network 506 is shown in Table IV.

TABLE IV

| Layer | Shape | Parameters |
|---|---|---|
| Input | 4 | 0 |
| Dense | 128 | 640 |
| Reshape | (8, 16) | 0 |
| Convolutional 1D Transpose | (16, 16) | 2576 |
| Convolutional 1D Transpose | (32, 16) | 2576 |
| Convolutional 1D Transpose | (64, 16) | 2576 |
| Convolutional 1D Transpose | (128, 8) | 1288 |
| Dense | (128, 1) | 9 |

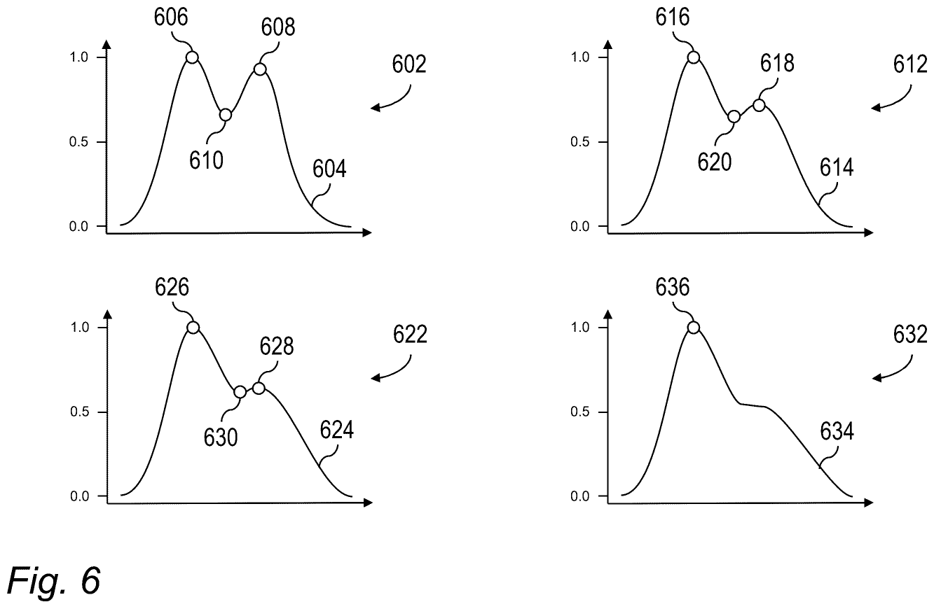

The encoder-decoder network 502 is trained on a dataset of over 1 million synthetic PPG pulses and approximately 6,000 annotated real pulses. As described in more detail below in relation to FIG. 6, the dataset of synthetic PPG pulses is generated according to a mixture of Gaussian model parameterized by the mean of the two gaussians, the standard deviation of the two gaussians, and a multiplicative factor for the amplitude of the first gaussian. In one implementation, the encoder-decoder network 502 is trained over 150 epochs using a minibatch stochastic gradient descent with a binary cross entropy loss function and a Kullback-Liebler divergence regularization term.

As stated above, in one embodiment, the encoder-decoder network 502 is a supervised VAE further comprising the fully connected layer 514 and the linear activation function 516. The fully connected layer 514 receives the latent vector 512 and may also receive the multimodal inputs 518. The linear activation function 516 is connected to the fully connected layer 514 to provide a value p∈[−1, +1]. The value p may thus be understood as a degree of “notchiness” of the pulse represented by the input vector (i.e., the extent/depth of the dicrotic notch within the pulse). This value may thus be used to determine whether or not a pulse extracted from a segment of pulse data is a valid or clean pulse. For example, a pulse having a p value greater than 0 may be determined as a valid pulse and thus used to determine blood pressure while a pulse having p value less than or equal to 0 may be discarded from further processing as an invalid or noisy pulse. Additionally, or alternatively, a pulse is also assessed based on the autoencoder's reconstruction loss; if the reconstruction loss value is above a threshold value, then the pulse is determined to be possibly affected by noise and is hence excluded from further processing.

FIG. 6 shows a set of synthetic pulses generated using a mixture of Gaussians model. Synthetic pulses such as those shown in FIG. 6 may be used to train a machine learning model to detect the systolic and diastolic peaks as well as the dicrotic notch. That is, the synthetic pulses may form a part of a training data set used to train an encoder-decoder network such as that shown in FIG. 5.

FIG. 6 shows a first plot 602 of a first synthetic pulse 604 comprising a systolic peak 606, a diastolic peak 608, and a dicrotic notch 610. A second plot 612 shows a second synthetic pulse 614 comprising a systolic peak 616, a diastolic peak 618, and a dicrotic notch 620. A third plot 622 shows a third synthetic pulse 624 comprising a systolic peak 626, a diastolic peak 628, and a dicrotic notch 630. A fourth plot 632 shows a fourth synthetic pulse 634 comprising a systolic peak 636.

Each plot shown in FIG. 6 illustrates a synthetic pulse generated according to a mixture of gaussians model that is parameterized by the mean of the two gaussians, the standard deviation of the two gaussians, and a multiplicative factor for the amplitude of the first gaussian. In the first plot 602, the multiplicative factor is such that the systolic peak 616 and the diastolic peak 618 are roughly the same amplitude. In consequence, the dicrotic notch 610 is well defined. In contrast, the multiplicative factor used for the fourth plot 632 is such that the diastolic peak and the dicrotic notch are not well defined.

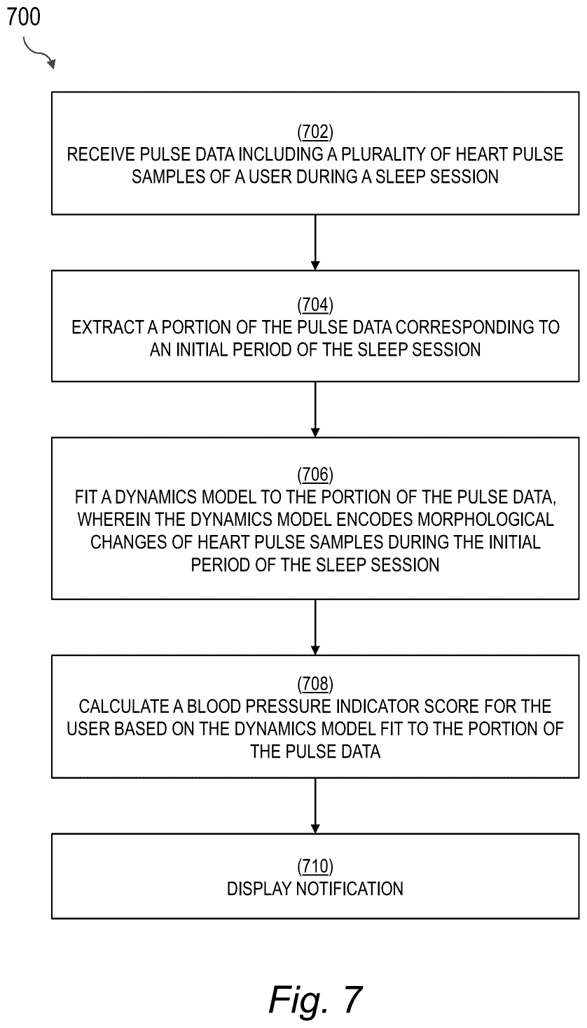

FIG. 7 shows a method 700 for calculating a blood pressure indicator score for a user based on pulse data obtained from the user by a wearable physiological monitor. The method 700 may be used in cooperation with any of the devices, systems, and methods described herein, such as by a user device (e.g., a mobile device) that is communicatively coupled to a wearable, continuous physiological monitoring device. For example, the method 700 may be used with the one or more user devices 1220 that are communicatively coupled to the physiological monitor 1206, as illustrated in FIG. 12.

As shown in step 702, the method 700 may include receiving, from a wearable physiological monitor worn by a user, pulse data including a plurality of heart pulse samples of the user during a sleep session. The pulse data may include a sequence of pulse segments (or segments of pulse data) obtained by the wearable physiological monitor at different time points during the sleep session. Each segment of pulse data may include a 20-40 second portion of pulse data comprising around 20-40 pulses or heart pulse samples. The plurality of heart pulse samples may be aggregated prior to step 704.

As shown in step 704, the method 700 may include extracting a portion of the pulse data corresponding to an initial period of the sleep session. The initial period of the sleep session may be a fixed period of time from the onset (start) of the sleep session. For example, the initial period of the sleep session may be the first 30 minutes, 60 minutes, 90 minutes, or the like of the sleep session.

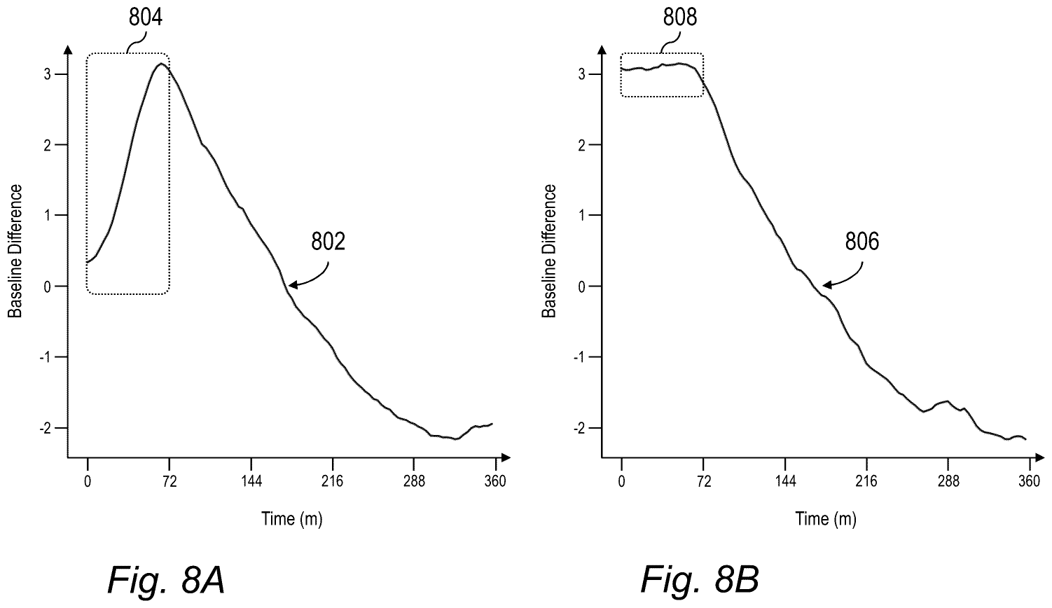

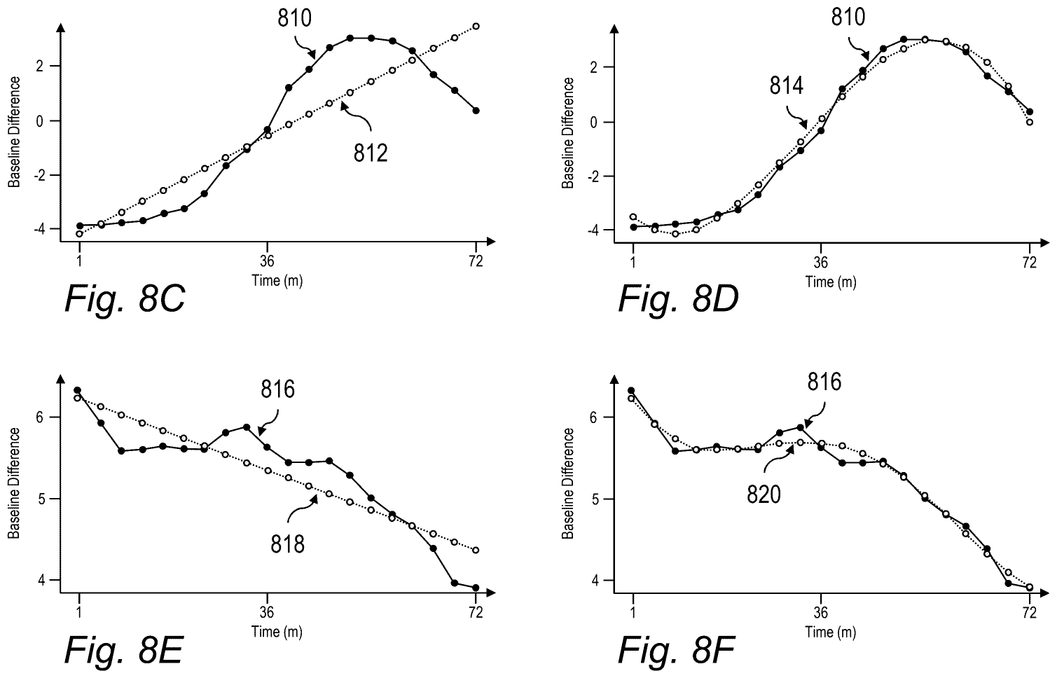

The portion of the pulse data may be transformed as part of step 704. The portion of the pulse data may be transformed to heart rate values such that the portion of the pulse data corresponds to heart rate values during the initial period of the sleep session. For example, the average heart rate may be calculated over a sequence of fixed subframes of the portion of the pulse data obtained at step 702 and recorded to form a sequence of average heart rate values. A baseline heart rate value (e.g., an average heart rate value over the sleep session) may be extracted from the sequence of heart rate values such that the portion of the pulse data corresponds to differences from baseline heart rate (as shown in FIGS. 8A and 8B).

As shown in step 706, the method 700 may include fitting a model to the portion of the pulse data. The model encodes dynamics of the portion of the pulse data during the initial period of the sleep session. When observing changes in the pulse data over an initial period of a sleep session, the dynamics of the changes differ for normal and hypotensive subjects than for hypertensive subjects (as shown in FIGS. 8A and 8B and described below). The model fit at step 706 captures this dynamical change (i.e., the changes in dynamics of the pulse data) thereby allowing differentiation between hypertensive and non-hypertensive subjects based on the difference in quantifiable dynamics of a blood pressure indicator. Here, dynamics and dynamical change may be understood as referring to the change over time of characteristics of the pulse data such as frequency (heart rate), morphology, or the like. In one example, the model, or dynamics model, encodes changes in heart rate during the initial period of the sleep session. The model may be a linear model (e.g., a linear regression model or the like) or a nonlinear model (e.g., a polynomial regression model, a spline based model, or the like).

As shown in step 708, the method 700 may include calculating a blood pressure indicator score for the user based on the model fit to the portion of the pulse data. As the model fit at step 706 captures dynamical changes in characteristics of the pulse data during the initial period of the sleep session, parameters or characteristics of the fit model may be used as a blood pressure indicator score for the user or as a feature used to estimate an indicator of baseline blood pressure. The blood pressure indicator score may be a hypertension score for the user (e.g., a probability or likelihood associated with the user having or exhibiting hypertension). The hypertension score may be calculated based on a trend in the model fit to the portion of the pulse data. For example, if the model is a linear model, then the hypertension score may be calculated based on a gradient of the linear model. More particularly, the hypertension score may be inversely correlated with the gradient of the linear model such that a linear model with a negative gradient indicates a high hypertension score while a linear model with a positive gradient indicates a low hypertension score (as shown in FIGS. 8C and 8E as described below). As a further example, if the model is a nonlinear model (e.g., a fourth-degree polynomial) then the hypertension score may be calculated based on one or more coefficients of the nonlinear model. As with the linear model, the hypertension score may be inversely correlated with the coefficients. For example, if the leading coefficient (e.g., the coefficient of the term having the highest exponent) is negative then this indicates a high hypertension score while if the leading coefficient is positive then this indicates a low hypertension score.

The features or characteristics extracted from the model fit at step 706 may be used as a further feature provided to a machine learning model (e.g., the one or more machine learning models 316 shown in FIG. 3A) to determine an indicator of baseline blood pressure. As such, the method 700 of FIG. 7 may be used in conjunction with the method 200 of FIG. 2A to determine an indicator of baseline blood pressure value based on the dynamic model features (e.g., trends of the fit model as described above), the resting heart rate, static feature(s), and/or dynamics feature(s) extracted from the portion of the pulse data. Demographic features may also be used. As such, the method 700 of FIG. 7 may be used to further improve the accuracy and robustness of the indicator of baseline blood pressure estimation method and system of FIGS. 2A and 3A.

As shown in step 710, the method 700 may include displaying, on a device of the user, a notification associated with the blood pressure indicator score the user. For example, the blood pressure indicator score may be a hypertension score (or probability of hypertension) and the notification may comprise the hypertension score such that the user is able to see the hypertension score and take action as appropriate. The notification may also, or instead, comprise an alert associated with the blood pressure indicator score. For example, the blood pressure indicator score may be a hypertension score, and the alert may be displayed when the user's hypertension score is above a predefined threshold thereby warning them of a potential change in their physiological condition. The alert may take the form of a visual alert displayed on a device (e.g., a user's personal or mobile device) or haptic feedback output by a device (e.g., haptic feedback provided by a physiological monitor).

FIGS. 8A-8F show an illustrative example of hypertension classification using the method 700 of FIG. 7. FIG. 8A shows a first waveform 802 and a first region 804. The first waveform 802 is associated with pulse data obtained from a first user during a sleep session. The first user is physiologically normal with respect to blood pressure (i.e., they are not hypertensive). The pulse data characterizes the cardiac activity of the first user during the sleep session and may be obtained from a wearable physiological monitor worn by the first user. More specifically, the first waveform 802 corresponds to a difference between the first user's heart rate—as determined by the pulse data—and their baseline heart rate during the sleep session. The first region 804 corresponds to a region of the first waveform 802 that spans an initial period of the sleep session (i.e., the first 72 minutes of the sleep session). FIG. 8B shows a second waveform 806 and a second region 808. The second waveform 806 is associated with pulse data obtained from a second user during a sleep session. The skilled person will appreciate that the sleep session of the first user and the sleep session of the second user need not be the same (i.e., they may not cover the same period of time). The pulse data characterizes the cardiac activity of the second user during their sleep session and may be obtained from a wearable physiological monitor worn by the second user. More specifically, the second waveform 806 corresponds to a difference between the second user's heart rate—as determined by the pulse data—and their baseline heart rate during the sleep session. The second region 808 corresponds to a region of the second waveform 806 that spans an initial period of the sleep session (i.e., the first 72 minutes of the sleep session).

FIGS. 8C-8F illustrate different models (or dynamics models) fit to the regions of the waveforms shown in FIGS. 8A and 8B. FIG. 8C shows a first portion 810 of the first waveform 802 shown in FIG. 8A (e.g., the portion of the first waveform 802 within the first region 804) and a first model 812 fit to the first portion 810. The first model 812 is a linear model. FIG. 8D shows the first portion 810 of the first waveform 802 shown in FIG. 8A and a second model 814 fit to the first portion 810. The second model 814 is a nonlinear model (a fourth-degree polynomial model). FIG. 8E shows a first portion 816 of the second waveform 806 shown in FIG. 8B (e.g., the portion of the second waveform 806 within the second region 808) and a first model 818 fit to the first portion 816. The first model 818 is a linear model. FIG. 8F shows the first portion 816 of the second waveform 806 shown in FIG. 8B and a second model 820 fit to the first portion 816. The second model 820 is a nonlinear model (a fourth-degree polynomial model).

As can be seen in FIGS. 8C-8F, both the linear and the nonlinear models encode the underlying dynamics of the pulse data during the initial portion of the sleep session (as represented by the waveforms). Moreover, the dynamical characteristics of the pulse data/waveforms differ between normal and hypertensive subjects. This is shown in the comparison between the models in FIGS. 8C and 8D (obtained from a normal subject) and the models in FIGS. 8E and 8F (obtained from a hypertensive subject). Therefore, fitting a dynamics model allows the differences in the change of pulse data over the initial period of a sleep session to be quantified and used as a hypertension score for the user. The features extracted from the dynamics model may be used directly to predict a hypertension score for the user or may be combined with one or more other features (e.g., resting heart rate, static features, dynamic features, demographic features) to predict an indicator of baseline blood pressure value for the user.

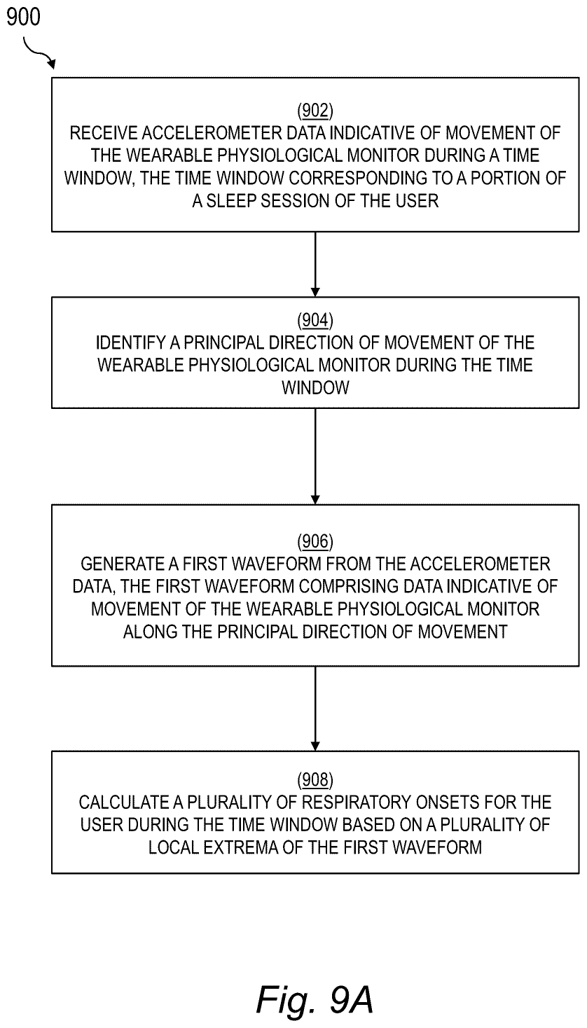

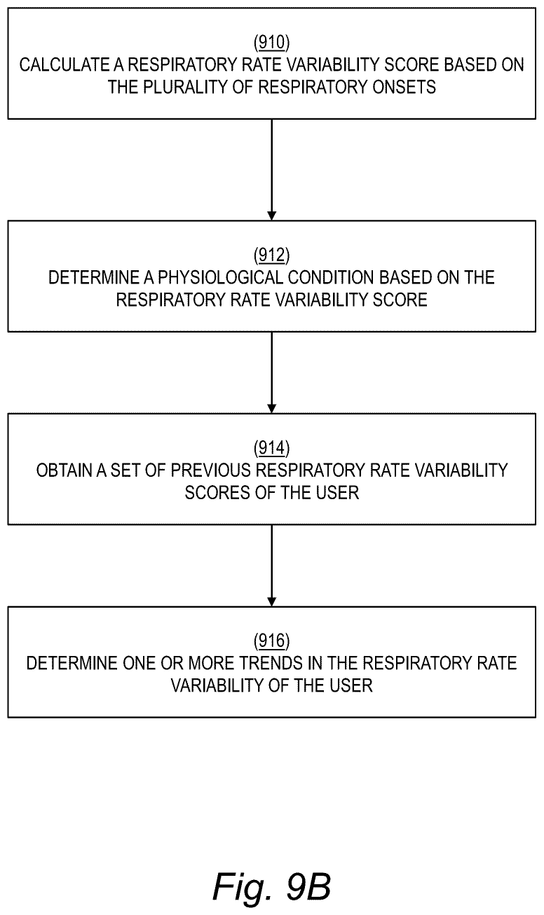

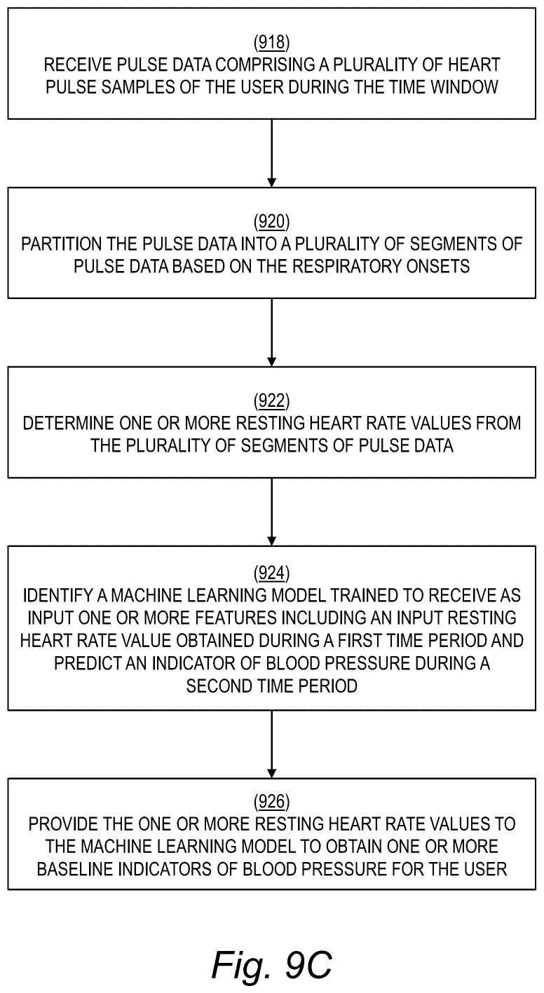

FIG. 9A is a flow chart illustrating a method 900 for respiratory onset estimation based on accelerometer data of a wearable physiological monitor. The method 900 may be used in cooperation with any of the devices, systems, and methods described herein, such as by a user device (e.g., a mobile device) that is communicatively coupled to a wearable, continuous physiological monitoring device. For example, the method 900 may be used with the one or more user devices 1220 that are communicatively coupled to the physiological monitor 1206, as illustrated in FIG. 12. In general, the method 900 calculates respiratory onsets (i.e., the onset of breathing cycles or the start of inhalation) for a user based on accelerometer data obtained from a wearable physiological monitor worn by the user during a period of sleep. Analyzing the movement of the wearable physiological monitor in a principal movement direction—e.g., a direction that is substantially aligned with the direction of gravity—allows the breathing pattern or respiratory cycles of the user to be recovered and used to determine respiratory onsets. Information or data pertaining to the respiratory cycles (or breathing cycles) of a user may be useful for diagnosis and monitoring of respiratory conditions, early detection of respiratory distress, and/or personalized care (e.g., helping aid user recovery, improve sleep, etc.). The method 900 provides a non-invasive technique for recovering information regarding a user's respiratory cycles from data that can be non-invasively obtained from a user's personal device, such as a wearable physiological device worn by the user. This opens up a rich vein of data to be generated from which insights into the user's physiological condition may be efficiently and effectively obtained. The user is thus provided with “at home” insights into their respiratory state and/or physiological condition without requiring specialized medical equipment or the performance of invasive techniques. This technique may be used alone, or may be used in combination with other respiratory properties such as respiratory sinus arrhythmia (based on cardiac data) to improve the measurement of respiratory cycles for a wearer of a physiological monitor.

As shown in step 902, the method 900 may include receiving, from a wearable physiological monitor worn by a user, accelerometer data indicative of movement of the wearable physiological monitor during a time window. More specifically, the wearable physiological monitor may be worn on a wrist of the user during the time window. Alternatively, the wearable physiological monitor may be worn on the chest of the user during the time window. Here, the time window corresponds to a portion of a sleep session of the user. The time window may be identified by identifying a period of low motion of the wearable physiological monitor during the time sleep session. Advantageously, focusing analysis on low motion regions helps ensure that the accelerometer data is not affected by motion noise that occurs due to the user's sleeping position or movement. That is, the movement of the wearable physiological monitor during a low motion period is primarily related to motion occurring due to the breathing activity of the user. Moreover, this allows for the method to work across a range of different sleep orientations of the user (e.g., sleeping with hands flat, sleeping with hands perpendicular, sleeping with hands on belly, sleeping on belly with hands flat, etc.).

A period of low motion may be identified by identifying a time period wherein an average motion of the wearable physiological monitor during the time period is below a threshold level of motion. For example, low motion regions may be identified by calculating the standard deviation across a moving window of 2,000 samples (20 packets) with a stride of one sample and looking for regions where the standard deviation drops below a threshold value (e.g., 0.02, 0.01, etc.). These regions are then annotated as the starting point of a low motion region. The point at which the standard deviation subsequently increases above the threshold value is annotated as the ending point of the low motion region. In this way, multiple potential low motion regions may be identified from accelerometer data of a single sleep session. The potential low motion regions may be further filtered by only maintaining regions that span at least 3,000 samples (300 packets).

As shown in step 904, the method 900 may include identifying, based on the accelerometer data, a principal direction of movement of the wearable physiological monitor during the time window. In general, the principal direction of movement of the wearable physiological monitor corresponds to the movement in the direction of gravity as it is the movement in this direction that most faithfully captures the user's breathing patterns. However, due to the sleeping position of the user, the principal direction of movement may not be in a single axis of the accelerometer data (e.g., the Z-axis). Therefore, the principal direction of movement may need to be extracted, or identified, from the accelerometer data.

The principal direction of movement may correspond to a component of maximum variance determined from the accelerometer data. That is, principal components analysis (PCA) may be applied to the accelerometer data and the principal component of maximum variance identified as the principal direction of movement of the wearable physiological monitor. Alternatively, the principal direction of movement may correspond to an axis of movement of the wearable physiological monitor having maximum mean absolute magnitude during the time window. That is, the principal direction of movement may correspond to one of the fixed axes of movement of the accelerometer (e.g., the X-axis, Y-axis, or Z-axis) that has maximum mean absolute magnitude during the time window. Alternatively, the principal direction of movement may be calculated as a dot product of representative accelerometer values along a plurality of axis of the accelerometer. That is, the mean value of the accelerometer for each of the three fixed axes (X, Y, Z) of the accelerometer is calculated and the dot product taken.

As shown in step 906, the method 900 may include generating a first waveform from the accelerometer data. The first waveform comprises data indicative of movement of the wearable physiological monitor along the principal direction of movement. If the principal direction of movement corresponds to one of the fixed axes of the accelerometer (e.g., the X-axis, Y-axis, or Z-axis), then the first waveform corresponds to the 1-dimensional accelerometer signal along that fixed axis. If the principal direction of movement is a component of the accelerometer data (e.g., a principal component), then the accelerometer data may be projected onto the component to generate a 1-dimensional signal comprising data indicative of movement of the wearable physiological monitor along the principal direction of movement. The accelerometer data, or the projection of the accelerometer data, may be interpolated to generate the first waveform (e.g., using cubic spline interpolation).

As shown in step 908, the method 900 may include calculating a plurality of respiratory onsets for the user during the time window based on a plurality of local extrema of the first waveform. More specifically, the first waveform may be inverted and a peak finding algorithm used to identify the peaks (local extrema/maxima) of the inverted waveform that correspond to the locations of inspiration (i.e., respiratory onsets). The time points associated with the local extrema thus correspond to the time points associated with the respiratory onsets of the user.

FIG. 9B shows additional steps that may be performed as part of the method 900 shown in FIG. 9A. That is, in some examples, the steps shown in FIG. 9B may be performed after the calculation of the plurality of respiratory onsets at step 908. In general, the additional steps shown in FIG. 9B utilize the respiratory onsets to calculate a respiratory rate variability score that can be linked to a physiological condition associated with the user.

As shown in step 910, the method 900 may include calculating a respiratory rate variability score based on a metric calculated using the plurality of respiratory onsets. Respiratory rate variability refers to the natural fluctuations in a user's rate of breathing over time. The respiratory rate variability (RRV) score quantifies the estimated respiratory rate variability of the user during the time window from the plurality of respiratory onsets. The RRV score may be calculated using one or more metrics and the plurality of respiratory onsets. In general, a metric (or RRV metric) returns a quantitative value, or score, that is indicative of, or associated with, the respiratory rate variability of the user during the time window.

The metric may be a standard deviation of respiratory cycle length metric. Respiratory cycle length (RCL) is the length (in time, e.g., seconds, milliseconds, etc.) of a single respiratory cycle. RCL may be calculated as the absolute difference between consecutive respiratory onsets and may be alternatively referred to as breath-to-breath interval (BBI). For example, if a first respiratory onset is identified at time point tₐ and the next respiratory onset is identified at time point t_{b}, then these two respiratory onsets bound a single respiratory cycle having an RCL of |tₐ−t_{b}|. The plurality of respiratory onsets determined at step 910 of the method 900 may therefore be used to generate one or more RCL values (referred to herein as the set of RCL values). The standard deviation of RCL length metric may be calculated as the standard deviation of the set of RCL values.

The metric may be a standard deviation of successive differences (SDSD) of RCL metric. Given two successive respiratory cycles with RCL values of RCL₁=|tₐ−t_{b}| and RCL₂=|t_{b}−t_{c}|, the successive difference between the two respiratory cycles is |RCL₁−RCL₂|. Given the set of RCL values (generated as described above), a set of successive differences of RCL values may then be calculated and the standard deviation of this set calculated as the SDSD of RCL metric. Advantageously, the SDSD metric helps capture local changes in the user's breathing pattern over the time window.

The metric may be a root mean square of successive differences (RMSSD) of RCL metric. The metric may be calculated by taking the square root of the mean value of the set of squared successive differences (calculated as described above).

The metric may be a coefficient of variation of RCL metric. The coefficient of variation is calculated by normalizing the standard deviation of the set of RCL values relative to the mean value of the set of RCL values. Advantageously, the coefficient of variation is a scale free measure that allows for comparison across individuals or populations with differing average RCL values.

The metric may be a median absolute deviation from median (MADM) RCL metric. This metric measures the variability of RCL values around the median RCL value. The MADM RCL metric may be calculated as the median of the absolute differences between the RCL values in the set of RCL values and the median value of the set of RCL values. Advantageously, the MADM RCL metric provides a robust measure of variability that is less sensitive to noise and outlier values.

The metric may be a coefficient of variation based on an absolute deviation from median RCL metric. This metric may be calculated by normalizing the MADM RCL metric relative to the median value of the set of RCL values.

As shown in step 912, the method 900 may include determining a physiological condition associated with the user based on the respiratory rate variability score. The respiratory rate variability score provides a quantitative and comparable representation of the respiratory state of the user during the time window and so may be used to determine a physiological condition associated with the user, such as a recovery state or a sleep stage.

In one example, a recovery state of the user may be determined from the RRV score. For example, based on the user's RRV score determined during a night's sleep, a recovery state may be determined and represented as a percentage (e.g., a recovery score where a lower percentage indicates that the user is still in a recovery state whereas a higher percentage indicates that the user is recovered and ready to take on exercise/strain). The recovery state may be directly based on the user's RRV; for example, a low RRV score may indicate a high level of recovery and a high RRV score may indicate a low level of recovery. Also, or instead, the recovery state may be determined as a weighted combination of the user's RRV score along with other physiological metrics such as heart rate variability, sleep score, and recent strain.

In one example, a sleep stage of the user may be determined from the RRV score using a sleep stage prediction model. The sleep stage prediction model may be rule based where the RRV score is compared against threshold values to determine sleep stage. For example, if the RRV is below a first threshold, then it is predicted that the user was/is in a stage 3 (deep sleep) non-REM sleep stage during the time window, and if the RRV is above the first threshold but below a second threshold then it is predicted that the user was/is in a stage 2 non-REM sleep stage during the time window. The sleep stage prediction model may be a machine learning model trained to map from RRV score to sleep stage. For example, a training data set of RRV scores and associated sleep stage labels (e.g., 0 for REM sleep, 1 for stage 1 non-REM sleep, 2 for stage 2 non-REM sleep, etc.) may be used to train a classifier to predict a sleep stage label from a given RRV score. The skilled person will appreciate that any suitable classifier may be used such as a decision tree classifier, a Random Forest classifier, a multilayer perceptron, and the like.

As shown in step 914, the method 900 may include obtaining a set of previous respiratory rate variability scores of the user. The set of previous RRV scores may have been previously calculated for the user over a range of time windows. The range of time windows may collectively represent a single event (e.g., a single night's sleep) or time windows over a longer time period (e.g., over 2 days, 4 days, 1 week, 1 month, etc.). The set of previous RRV scores may be obtained from a storage location such as a memory or persistent memory of a device (e.g., the one or more devices 1220 shown in FIG. 12) and may be transmitted to the device or server performing the method 900.

As shown in step 916, the method 900 may include determining one or more trends in the respiratory rate variability of the user. The set of previous RRV scores may be compared, analyzed, or otherwise processed to determine one or more trends or changes in the user's RRV. For example, the set of RRV scores obtained for a user over a single night's sleep may be analyzed to identify specific times or periods in which the user reached a certain sleep stage (e.g., when they were in deep sleep and for how long). As a further example, the set of RRV scores obtained for a user over a period of 1 month may be analyzed to identify a change in the RRV, recovery rate, or the like of the user of the 1 month period. Such trends provide the user with non-invasive feedback into their longitudinal physiological condition/state that can aid in their recovery and/or help drive longer term health improvements.

A notification based on the physiological condition may then be output. The notification may include information based on the physiological condition, the RRV score, and/or the respiratory onsets. For example, the notification may take the form of a notification, prompt, or information screen displayed on a device of the user (e.g., the one or more devices 1220 shown in FIG. 12) and including the information (e.g., “your recovery rate is XX %”). In one example, the notification includes information based on the one or more trends in the RRV of the user. For example, the notification may include information detailing sleep stage information for the user over one or more nights of sleep. As a further example, the notification may include a graph or chart of recovery rate of the user over a time period (e.g., 2 days, 4 days, 1 week, etc.).

The notification may also take the form of haptic feedback provided to the user via a device (e.g., the one or more devices 1220 or the wearable physiological monitor 1206 shown in FIG. 12). The notification may be provided to the user if one or more conditions based on the RRV score are met. For example, if the user's recovery rate is above a threshold level then haptic feedback may be provided to the user to indicate that their threshold recovery rate has been reached.

FIG. 9C shows additional steps that may be performed as part of the method 900 shown in FIG. 9A. That is, in some examples, the steps shown in FIG. 9C may be performed after the calculation of the plurality of respiratory onsets at step 908. The steps shown in FIG. 9C may also be performed in conjunction with or after the steps shown in FIG. 9B. In general, the additional steps shown in FIG. 9C utilize the respiratory onsets to segment a sequence of pulse data that is substantially temporally aligned with the accelerometer data obtained at step 902. The segmented pulse data may then be used to determine an indicator of baseline blood pressure for the user (as described in relation to FIGS. 1A and 2A above) or an aggregated indicator of baseline blood pressure for the user (as described in relation to FIGS. 1B and 2B above).

As shown in step 918, the method 900 may include receiving, from the wearable physiological monitor, pulse data comprising a plurality of heart pulse samples of the user during the time window. The pulse data may be temporally aligned, or substantially temporally aligned, with the accelerometer data obtained at step 902.

As shown in step 920, the method 900 may include partitioning the pulse data into a plurality of segments of pulse data based on the respiratory onsets. The plurality of respiratory onsets are associated with a corresponding plurality of time points. The time points may be used as boundary points for segmenting the pulse data such that the extracted segments comprise heart pulse samples that occur during a single respiratory cycle of the user. For example, if a first respiratory onset is identified at time point tₐ and a subsequent respiratory onset identified at time point t_{b}, then a segment of pulse data may be extracted from the pulse data by extracting the pulse data spanning the time period tₐ→t_{b}. The extracted segments may then be used in any of the analysis approaches of the present disclosure (e.g., as described in relation to FIGS. 1A-8F above) to help improve the accuracy and robustness of the estimated baseline indicators of blood pressure.

As shown in step 922, the method 900 may include determining, from a first segment of the plurality of segments of pulse data, a first resting heart rate value of the user during the time window. Step 922 may correspond to step 104 of the method 100 described above in relation to FIG. 1A.

As shown in step 924, the method 900 may include identifying a machine learning model trained to receive as input one or more features including an input resting heart rate value obtained during nighttime and predict an indicator of blood pressure during daytime. Step 924 may correspond to step 106 of the method 100 described above in relation to FIG. 1A.

As shown in step 926, the method 900 may include providing the first resting heart rate value to the machine learning model to obtain a first indicator of baseline blood pressure for the user. Step 926 may correspond to step 108 of the method 100 described above in relation to FIG. 1A.

While steps 922-926 of FIG. 9C illustrate the use of the partitioned pulse data in relation to the method 100 of FIG. 1A, the segmented pulse data may also or instead be used as input to the methods 200 and/or 216 described above in relation to FIGS. 2A and 2B respectively.

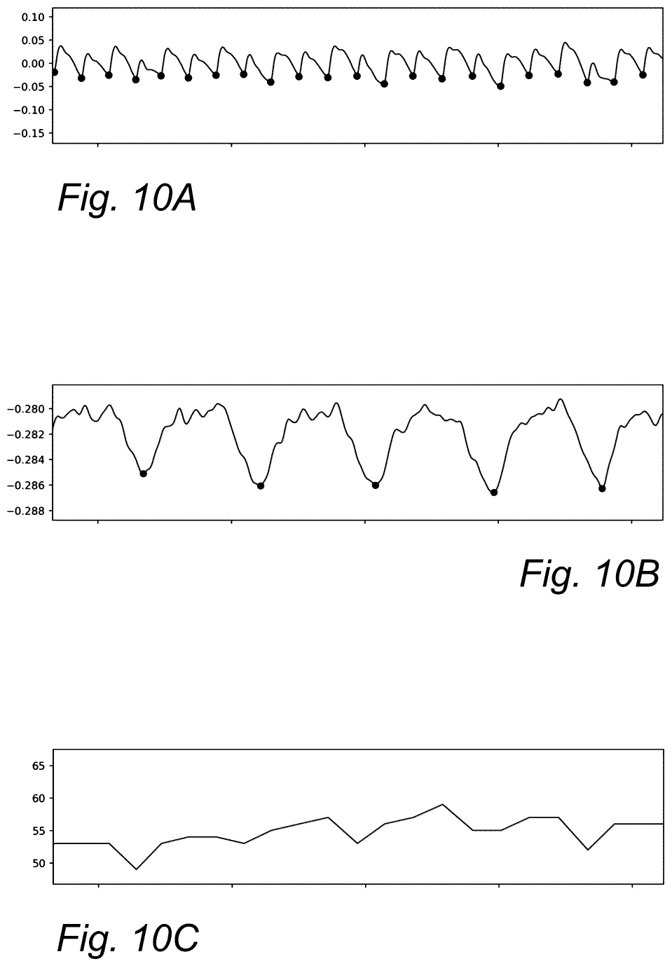

FIGS. 10A-10C illustrate accelerometer based respiratory onset detection.

FIG. 10A shows a waveform corresponding to the pulse data of a user during a portion of a sleep session. The filled black circles shown in FIG. 10A correspond to boundary points (e.g., start and end points) of individual heart pulse samples or heartbeats. FIG. 10B shows a respiratory cycle waveform recovered from accelerometer data of a wearable physiological monitor using the method 900 shown in FIG. 9A. The filled black circles shown in FIG. 10B correspond to the respiratory onsets identified from the waveform. FIG. 10C shows the ground truth R-R intervals obtained over the portion of the sleep session. It is known that the R-R interval reduces with inspiration and increases with exhalation. As can be seen in the comparison between FIGS. 10B and 10C, the respiratory onsets identified from the accelerometer data substantially aligns temporally with the local minima of the R-R interval data (i.e., the inhalation points).

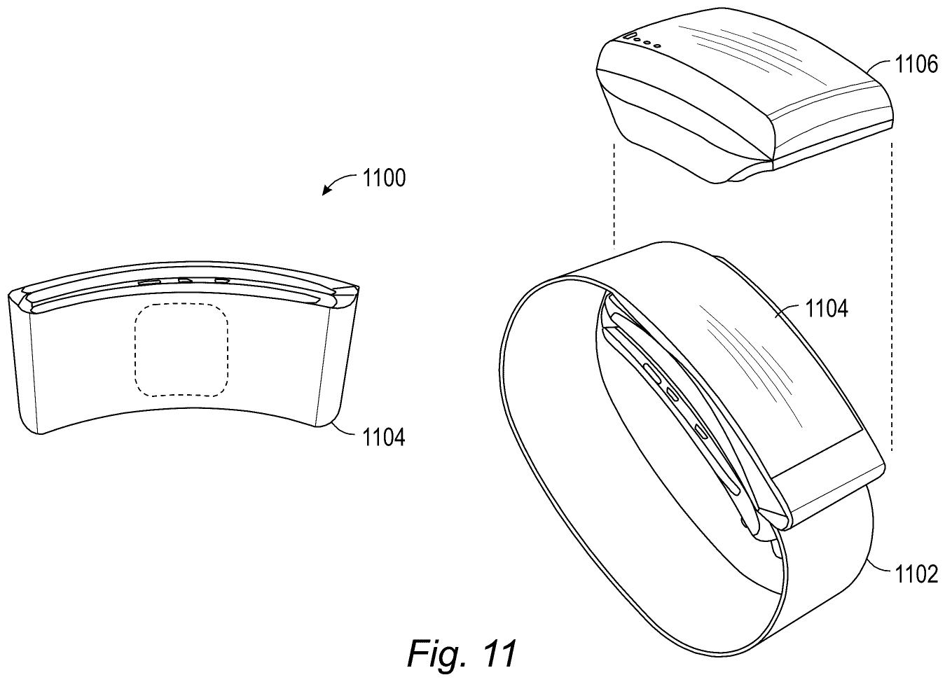

FIG. 11 shows a physiological monitoring device. The overall system 1100 may include a device 1104 (which may or may not include a display screen or other user interface) generally configured for physiological monitoring. The system 1100 may further include a removable and replaceable battery 1106 for recharging the device 1104. A strap 1102 may be provided, and may include any arrangement suitable for retaining the device 1104 in a position on a wearer's body for acquisition of physiological data as described herein. For example, the strap 1102 may include slim elastic band formed of any suitable elastic material, for example, a rubber, a woven polymer fiber such as a woven polyester, polypropylene, nylon, spandex, and so forth. The strap 1102 may be adjustable to accommodate different wrist sizes, and may include any latches, hasps, or the like to secure the device 1104 in an intended position for monitoring a physiological signal. While a wrist-worn device is depicted, it will be understood that the device 1104 may be configured for positioning in any suitable location on a user's body, based on the sensing modality and the nature of the signal to be acquired. For example, the device 1104 may be configured for use on a wrist, an ankle, a bicep, a chest, or any other suitable location(s), and the strap 1102 may be, or may include, a waistband or other elastic band or the like within an article of clothing or accessory. The device 1104 may also or instead be structurally configured for placement on or within a garment, e.g., permanently or in a removable and replaceable manner. To that end, the device 1104 may be structurally configured for placement within a pocket, slot, and/or other housing that is coupled to or embedded within a garment. In such configurations, the garment may include sensing windows or other pathways such that the device 1104 can sense physiological and/or biomechanical parameters from a user wearing a garment that includes the device 1104 therein or thereon.

The system 1100 may include any hardware components, subsystems, and the like to provide various functions such as data collection, processing, display, and communications with external resources. For example, the system 1100 may include a heart rate monitor using, e.g., photoplethysmography, electrocardiogramy other technique(s). The system 1100 may be configured such that, when placed for use about a wrist, the system 1100 initiates acquisition of physiological data from the wearer. In some embodiments, the pulse or heart rate may be taken using an optical sensor coupled with one or more light emitting diodes (LEDs), all directly in contact with the user's wrist. The LEDs may be positioned to direct illumination toward the user's skin, and may be accompanied by one or more photodiodes or other photodetectors suitable for measuring illumination from the LEDs that is reflected and/or transmitted by the wearer's skin.

The system 1100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure, and the like, as well environmental or contextual parameters such as ambient light, ambient temperature, humidity, time of day, and the like. The system 1100 may also include other sensors such as accelerometers and/or gyroscopes for motion detection, and sensors for environmental temperature sensing, electrodermal activity (EDA) sensing, galvanic skin response (GSR) sensing, and the like.

The system 1100 may include one or more sources of battery life, such as a first battery environmentally sealed within the device 1104 and a battery 1106 that is removable and replaceable to recharge the battery in the device 1104. Also or instead, the system 1100 may include a plurality of devices 1104, where such devices 1104 may be able to provide power to one another. The system 1100 may perform numerous functions related to continuous monitoring, such as automatically detecting when the user is asleep, awake, exercising, and so forth, and such detections may be performed locally at the device 1104 or at a remote service coupled in a communicating relationship with the device 1104 and receiving data therefrom. In general, the system 1100 may support continuous, independent monitoring of a physiological signal such as a heart rate, and acquired data may be stored on the device 1104 until it can be uploaded to a remote processing resource for more computationally expensive analysis.

FIG. 12 illustrates a physiological monitoring system. More specifically, FIG. 12 illustrates a physiological monitoring system 1200 that may be used with any of the methods or devices described herein. In general, the system 1200 may include a physiological monitor 1206, a user device 1220, a remote server 1230 with a remote data processing resource (such as any of the processors or processing resources described herein), and one or more other resources 1250, all of which may be interconnected through a data network 1202.

The data network 1202 may be any of the data networks described herein. For example, the data network 1202 may be any network(s) or internetwork(s) suitable for communicating data and information among participants in the system 1200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 1200. This may also include local or short range communications networks suitable, e.g., for coupling the physiological monitor 1206 to the user device 1220, or otherwise communicating with local resources.

The physiological monitor 1206 may, in general, be any physiological monitoring device, such as any of the wearable monitors or other monitoring devices described herein. Thus, the physiological monitor 1206 may generally be shaped and sized to be worn on a wrist or other body location and retained in a desired orientation relative to the appendage with a strap 1210 or other attachment mechanism. The physiological monitor 1206 may include a wearable housing 1211, a network interface 1212, one or more sensors 1214, one or more light sources 1215, a processor 1216, a haptic device 1217 (and/or any other type of component suitable for providing haptic or other sensory alerts to a user), a memory 1218, and a wearable strap 1210 for retaining the physiological monitor 1206 in a desired location on a user.

In general, the physiological monitor 1206 may include a wearable physiological monitor configured to acquire heart rate data and/or other physiological data from a wearer. More specifically, the wearable housing 1211 of the physiological monitor 1206 may be configured such that a user can acquire heart rate data and/or other physiological data from the user in a substantially continuous manner. The wearable housing 1211 may be configured for cooperation with a strap 1210 or the like, e.g., for engagement with an appendage of a user.

The network interface 1212 may be configured to coupled one or more participants of the system 1200 in a communicating relationship, e.g., with the remote server 1230, either directly, e.g., through a cellular data connection or the like, or indirectly through a short range wireless communications channel coupling the physiological monitor 1206 locally to a wireless access point, router, computer, laptop, tablet, cellular phone, or other device that can relay data from the physiological monitor 1206 to the remote server 1230 as necessary or helpful for acquiring and processing data.

The one or more sensors 1214 may include any of the sensors described herein, or any other sensors suitable for physiological monitoring. By way of example and not limitation, the one or more sensors 1214 may include one or more of a light source, an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, a temporal sensor, an electrodermal activity sensor, and the like. The one or more sensors 1214 may be disposed in the wearable housing 1211, or otherwise positioned and configured for capture of data for physiological monitoring of a user. In one aspect, the one or more sensors 1214 include a light detector configured to provide data to the processor 1216 for calculating a heart rate variability. The one or more sensors 1214 may also or instead include an accelerometer configured to provide data to the processor 1216, e.g., for detecting activities such as a sleep state, a resting state, a waking event, exercise, and/or other user activity. In an implementation, the one or more sensors 1214 measure a galvanic skin response of the user.

The processor 1216 and memory 1218 may be any of the processors and memories described herein, and may be suitable for deployment in a physiological monitoring device. In one aspect, the memory 1218 may store physiological data obtained by monitoring a user with the one or more sensors 1214. The processor 1216 may be configured to obtain heart rate data from the user based on the data from the sensors 1214. The processor 1216 may be further configured to assist in a determination of a condition of the user, such as whether the user has an infection or other condition of interest as described herein.

The one or more light sources 1215 may be coupled to the wearable housing 1211 and controlled by the processor 1216. At least one of the light sources 1215 may be directed toward the skin of a user's appendage. Light from the light source 1215 may be detected by the one or more sensors 1214.

The system 1200 may further include a remote data processing resource executing on a remote server 1230. The remote data processing resource may be any of the processors described herein, and may be configured to receive data transmitted from the memory 1218 of the physiological monitor 1206, and to process the data to detect or infer physiological signals of interest such as heart rate, heart rate variability, respiratory rate, pulse oxygen, blood pressure, and so forth. The remote server 1230 may also or instead evaluate a condition of the user such as a recovery state, sleep quality, daily activity strain, and any health conditions that might be detected based on such data.

The system 1200 may also include one or more user devices 1220, which may work together with the physiological monitor 1206, e.g., to provide a display for user data and analysis, and/or to provide a communications bridge from the network interface 1212 of the physiological monitor 1206 to the data network 1202 and the remote server 1230. For example, physiological monitor 1206 may communicate locally with a user device 1220, such as a smartphone of a user, via short-range communications, e.g., Bluetooth, or the like, e.g., for the exchange of data between the physiological monitor 1206 and the user device 1220, and the user device 1220 may communicate with the remote server 1230 via the data network 1202. Computationally intensive processing, such as infection monitoring, may be performed at the remote server 1230, which may have greater memory capabilities and processing power than the physiological monitor 1206 that acquires the data.

The user device 1220 may include any computing device as described herein, including without limitation a smartphone, a desktop computer, a laptop computer, a network computer, a tablet, a mobile device, a portable digital assistant, a cellular phone, a portable media or entertainment device, and so on. The user device 1220 may provide a user interface 1222 for access to data and analysis by a user, and/or to control operation of the physiological monitor 1206. The user interface 1222 may be maintained by a locally-executing application on the user device 1220, or the user interface 1222 may be remotely served and presented on the user device 1220, e.g., from the remote server 1230 or the one or more other resources 1250.

In general, the remote server 1230 may include data storage, a network interface, and/or other processing circuitry. The remote server 1230 may process data from the physiological monitor 1206 and perform infection monitoring/analyses or any of the other analyses described herein, and may host a user interface for remote access to this data, e.g., from the user device 1220. The remote server 1230 may include a web server or other programmatic front end that facilitates web-based access by the user devices 1220 or the physiological monitor 1206 to the capabilities of the remote server 1230 or other components of the system 1200.

The other resources 1250 may include any resources that can be usefully employed in the devices, systems, and methods as described herein. For example, these other resources 1250 may include without limitation other data networks, human actors (e.g., programmers, researchers, annotators, editors, analysts, and so forth), sensors (e.g., audio or visual sensors), data mining tools, computational tools, data monitoring tools, algorithms, and so forth. The other resources 1250 may also or instead include any other software or hardware resources that may be usefully employed in the networked applications as contemplated herein. For example, the other resources 1250 may include payment processing servers or platforms used to authorize payment for access, content, or option/feature purchases, or otherwise. In another aspect, the other resources 1250 may include certificate servers or other security resources for third-party verification of identity, encryption or decryption of data, and so forth. In another aspect, the other resources 1250 may include a desktop computer or the like co-located (e.g., on the same local area network with, or directly coupled to through a serial or USB cable) with a user device 1220, wearable strap 1210, or remote server 1230. In this case, the other resources 1250 may provide supplemental functions for components of the system 1200.

The other resources 1250 may also or instead include one or more web servers that provide web-based access to and from any of the other participants in the system 1200. While depicted as a separate network entity, it will be readily appreciated that the other resources 1250 (e.g., a web server) may also or instead be logically and/or physically associated with one of the other devices described herein, and may for example, include or provide a user interface 1222 for web access to a remote server 1230 or a database in a manner that permits user interaction through the data network 1202, e.g., from the physiological monitor 1206 or the user device 1220, with processing and data resources of the remote server 1230.

One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issues, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrocardiogram (ECG) based heart rate measurements to be gathered from a torso region of the wearer; wireless antennas to be placed above the upper portion of the thoracic spine to achieve desired communications signals; a contactless payment system to be embedded in a sleeve cuff for interactions with a payment terminal; and muscle oxygen saturation measurements to be gathered from muscles such as the pectoralis major, latissimus dorsi, biceps brachii, and other major muscle groups. This non-exhaustive list illustrates just some examples of technology that may be incorporated into a single garment.

Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment the incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

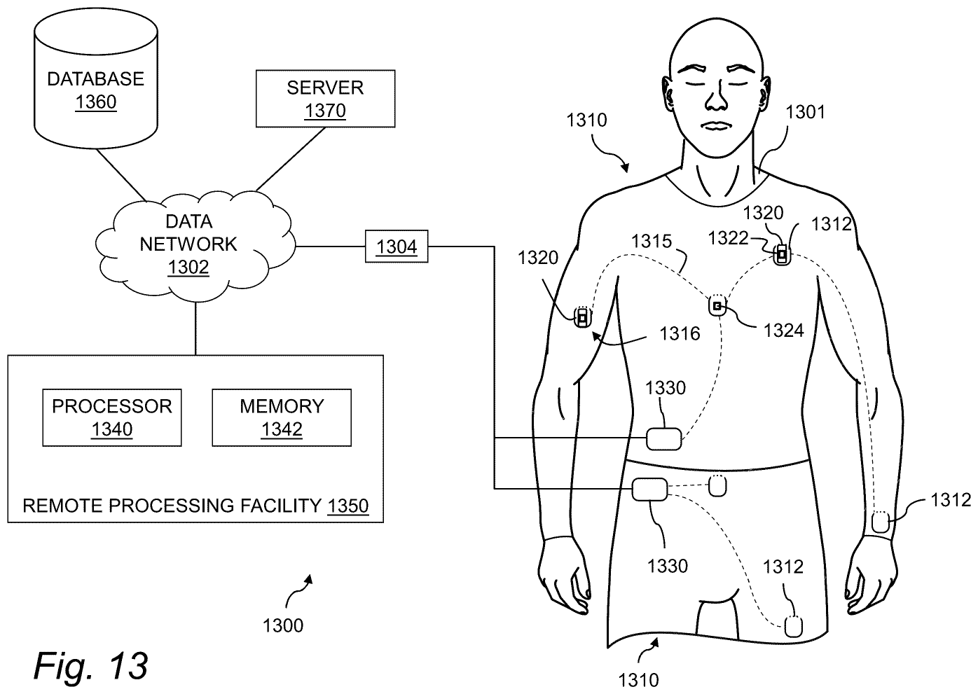

FIG. 13 shows a smart garment system. In general, the system 1300 may include a plurality of components—e.g., a garment 1310, one or more modules 1320, a controller 1330, a processor 1340, a memory 1342, and so on—capable of communicating with one another over a data network 1302. The garment 1310 may be wearable by a user 1301 and configured to communicate with a module 1320 having a physiological sensor 1322 that is structurally configured to sense a physiological parameter of the user 1301. As discussed herein, the module 1320 may be controllable by the controller 1330 based at least in part on a location 1316 where the module 1320 is located on or within the garment 1310. This position-based information may be derived from an interaction and/or communication between the module 1320 and the garment 1310 using various techniques. It will be understood that, while two controllers 1330 are shown, the garment 1310 may include a single inter-garment controller, or any number of separate controllers 1330 in any number of garments 1310 (e.g., one per garment, or one for all garments worn by a person, etc.), and/or controllers may be integrated into other modules 1320.

For communication over the data network 1302, the system 1300 may include a network interface 1304, which may be integrated into the garment 1310, included in the controller 1330, or in some other module or component of the system 1300, or some combination of these. The network interface 1304 may generally include any combination of hardware and software configured to wirelessly communicate data to remote resources. For example, the network interface 1304 may use a local connection to a laptop, smart phone, or the like that couples, in turn, to a wide area network for accessing, e.g., web-based or other network-accessible resources. The network interface 1304 may also or instead be configured to couple to a local access point such as a router or wireless access point for connecting to the data network 1302. In another aspect, the network interface 1304 may be a cellular communications data connection for direct, wireless connection to a cellular network or the like.

The data network 1302 may generally include any communication network through which computer systems may exchange data. For example, the data network 1302 may include, but is not limited to, the Internet, an intranet, a LAN (Local Area Network), a WAN (Wide Area Network), a MAN (Metropolitan Area Network), a wireless network, a cellular data network, an optical network, and the like. To exchange data via the data network 1302, the system 1300 and the data network 1302 may use various methods, protocols, and standards including, but not limited to, token ring, Ethernet, wireless Ethernet, Bluetooth, TCP/IP, UDP, HTTP, FTP, SNMP, SMS, MMS, SS7, JSON, XML, REST, SOAP, CORBA, IIOP, RMI, DCOM and Web Services. To ensure data transfer is secure, the system 1300 may transmit data via the data network 1302 using a variety of security measures including, but not limited to, TSL, SSL and VPN. By way of example, some embodiments of the system 1300 may be configured to stream information wirelessly to a social network, a data center, a cloud service, and so forth.

In some embodiments, data streamed from the system 1300 to the data network 1302 may be accessed by the user 1301 (or other users) via a website. The network interface 1304 may thus be configured such that data collected by the system 1300 is streamed wirelessly to a remote processing facility 1350, database 1360, and/or server 1370 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 1300 may include a cellular system or other hardware for independently accessing network resources from the garment 1310 without requiring local network connectivity.

In one example, the network interface 1304 may be configured to stream data using Bluetooth or Bluetooth Low Energy technology, e.g., to a nearby device such as a cell phone or tablet for forwarding to other resources on the data network 1302. In another example, the network interface 1304 may be configured to stream data using a cellular data service, such as via a 3G, 4G, or 5G cellular network. It will be understood that the network interface 1304 may include a computing device such as a mobile phone or the like. The network interface 1304 may also or instead include or be included on another component of the system 1300, or some combination of these. Where battery power or communications resources can advantageously be conserved, the system 1300 may preferentially use local networking resources when available, and reserve cellular communications for situations where a data storage capacity of the garment 1310 is reaching capacity. Thus, for example, the garment 1310 may store data locally up to some predetermined threshold for local data storage, below which data is transmitted over local networks when available. The garment 1310 may also transmit data to a central resource using a cellular data network only when local storage of data exceeds the predetermined threshold.

The garment 1310 may include one or more of a shirt (or other top), shorts/pants (or other bottom), an undergarment (e.g., undershirt, underwear, brassiere, and so on), a sock or other footwear, a shoe, a facemask, a hat or helmet (or other head adornment), a compression sleeve, a sweatband, kinesiology tape or clastic therapeutic tape, a glove, and the like. More generally, the garment 1310 may include any type(s) of wearable clothing or adornment suitable for wearing by a user and retaining one or more sensing modules as contemplated herein.

The garment 1310 may include one or more designated areas 1312 for positioning a module to sense a physiological parameter of the user 1301 wearing the garment 1310. One or more of the designated areas 1312 may be specifically tailored for receiving a module 1320 therein or thereon. For example, a designated area 1312 may include a pocket structurally configured to receive a module 1320 therein. Also or instead, a designated area 1312 may include a first fastener configured to cooperate with a second fastener disposed on a module 1320. One or more of the first fastener and the second fastener may include at least one of a hook-and-loop fastener, a button, a clamp, a clip, a snap, a projection, and a void.

The designated areas 1312 may include at least one of a torso region, a spinal region, an extremity region (e.g., one or more of an arm region such as a sleeve, and a leg region such as a pant leg), a waistband region, a cuff region, and so on. Also or instead, one or more of the designated areas 1312 may include at least a region adjacent to one or more muscle groups of the user 1301—e.g., muscle groups including at least one of the pectoralis major, latissimus dorsi, biceps brachii, and so on.

By placing a pocket or the like in one of these designated areas 1312, a position of a module 1320 can be controlled, and where an RFID tag, sensor, or the like is used, the designated area 1312 can specifically sense when a module 1320 is positioned there for monitoring, and can communicate the detected location to any suitable control circuitry. In this manner, a garment 1310 may facilitate the installation of modules 1320 in many different, discrete locations, the placement of which can be controlled by the configuration of the garment 1310, and the use of which can be automatically detected when corresponding control modules 1320 are placed there for use. Also or instead, the garment 1310 may facilitate the placing of the modules 1320 over relatively large regions of the garment 1310. For example, a garment 1310 may include a relatively large region (in terms of surface area) where a module 1320 can be affixed or otherwise secured, e.g., by loops, straps, buttons, sheets of hook-and-loop fasteners, and so forth.

In general, each designated area 1312 may include a pocket such as any of those described above, or any other mounting fixture or combination of fixtures. Where a pocket is used, the pocket may be configured as described above to preferentially urge a module 1320 within the pocket toward the user's skin under normal pressure. Without limiting the generality of the foregoing, this may generally include an exterior layer of the pocket that is less elastic than an interior surface of the pocket so that when circumferential tension is applied (e.g., when the garment 1310 is donned), the pocket preferentially urges a contact surface of the sensor inward toward the intended target surface with at least a predetermined normal force (when the garment 1310 is properly sized for the user). In this respect, it will be understood that although some variation in normal force among users and garments is inevitable, typical tensions for comfortable use of properly fitted athletic wear are generally known, and adequate contact force to obtain a high quality physiological signal is generally known, and in any event readily observable in acquired data. As such, adequate circumferential tensions and resulting normal contact forces needed to promote good contact between sensing regions of the module 1320 (such as LEDs, capacitive touch sensors, photodiodes, and the like) and the user's skin may readily be determined, and can advantageously facilitate the use of wrist-worn sensor housings such as those described above with one of the garments 1310 described herein for off-wrist monitoring if/when desired.

In one aspect, the designated areas 1312 may usefully be positioned where reinforcing elastic bands are typically provided on garments, e.g., around the mid-torso for a sports bra, around the waist on shorts or underwear, or on the sleeves of a t-shirt. In one aspect, the designated areas 1312 may also usefully be positioned according to the intended physiological measurement, e.g., near major arteries suitable for heart rate detection using photoplethysmography. In one aspect, the garment 1310 may usefully distribute these designated areas 1312 (and supporting infrastructure such as wired connectors, location identification tags, and the like) at the intersection of regions where good physiological signals can be obtained and regions where adequate normal forces for good sensor contact can be generated by clothing. For example, this may include the ankles, the waist, the mid-torso, the biceps, the wrists, the forehead, and so on.

The garment 1310 may also or instead incorporate other infrastructure 1315 to cooperate with a module 1320. For example, the garment infrastructure 1315 may include wires or the like embedded in the garment 1310 to facilitate wired data or power transfer between installed modules 1320 and other system components (including other modules 1320). The infrastructure 1315 may also or instead include integrated features for, e.g., powering modules, supporting data communications among modules, and otherwise supporting operation of the system 1300. The infrastructure may also or instead include location or identification tags or hardware, a power supply for powering modules 1320 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 1320, and so forth. More generally, any hardware, software, or combination of these suitable for augmenting operation of the garment 1310 and a physiological monitoring system using the garment 1310 may be incorporated as infrastructure 1315 into the garment 1310 as contemplated herein.

The modules 1320 may generally be sized and shaped for placement on or within the one or more designated areas 1312 of the garment 1310. For example, in certain implementations, one or more of the modules 1320 may be permanently affixed on or within the garment 1310. In such instances, the modules 1320 may be washable. Also or instead, in certain implementations, one or more of the modules 1320 may be removable and replaceable relative to the garment 1310. In such instances, the modules 1320 need not be washable, although a module 1320 may be designed to be washable and/or otherwise durable enough to withstand a prolonged period of engagement with a designated area 1312 of the garment 1310. A module 1320 may be capable of being positioned in more than one of the designated areas 1312 of the garment 1310. That is, one or more of the plurality of modules 1320 may be configured to sense data using a physiological sensor 1322 in a plurality of designated areas 1312 of the garment 1310.

Removable and replaceable modules 1320 may provide several advantages such as case of garment care (e.g., washing) and power management (e.g., removal for recharging). Furthermore, removability may facilitate replacement and/or repositioning of modules within the garment 1310 for different sensing activities or other reconfigurations, replacement of damaged or defective modules 1320, and so forth.

A module 1320 may include one or more physiological sensors 1322 and a communications interface 1324 programmed to transmit data from at least one of the physiological sensors 1322. For example, the physiological sensors 1322 may include one or more of a heart rate monitor, an oxygen monitor (e.g., a pulse oximeter), a thermometer, an accelerometer, a gyroscope, a position sensor, a Global Positioning System, a clock, a galvanic skin response (GSR) sensor, or any other electrical, acoustic, optical, or other sensor or combination of sensors and the like useful for physiological monitoring, environmental monitoring, or other monitoring as described herein. In one aspect, the physiological sensors 1322 may include a conductivity sensor or the like used for electromyography, electrocardiography, electroencephalography, or other physiological sensing based on electrical signals. The data received from the physiological sensors 1322 may include at least one of heart rate data, muscle oxygen saturation data, temperature data, movement data, position/location data, environmental data, temporal data, and so on.

In one aspect, a module 1320 may be configured for use on multiple body locations. For example, the module 1320 may be one of the wrist-worn sensors described above. The module 1320 may be adapted for use with a garment 1310 in various ways. In one aspect, the module 1320 may have relatively smooth, continuous exterior surfaces to facilitate sliding into and out of a pocket, such as any of the pockets described herein, or any other suitable retaining structure(s). In another aspect, an LED and/or sensor region may protrude from a surface of the module 1320 sufficiently to extend beyond a restraining garment material and into a contact surface of a user. The module 1320 may also include hardware to facilitate such uses. For example, a module 1320 may usefully incorporate a contact sensor for detecting contact with a user. However, the exposed contact surfaces of the module 1320 may be different when retained by a wrist strap (or other limb strap) than when retained by a garment pocket. To facilitate multiple retaining modes, the module 1320 may usefully incorporate two or more contact sensors (such as capacitive sensors or other touch sensors, switches, or the like) at two different locations, each positioned to detect contact with a wearer in a different retaining mode. For example, a module 1320 may include a capacitive sensor adjacent to an optical sensing system that contacts the user's skin when the module 1320 is retained with a wrist strap. The module 1320 may also or instead optically detect contact when the capacitive sensor is covered by a garment fabric or the like that prevents direct skin contact, or a second capacitive sensor may be placed within another region exposed by the garment 1310 retaining system. In another aspect, the garment 1310 may include a capacitive sensor that provides a signal to the module 1320, or to some other system controller or the like, when a region of the garment near the module 1320 is in contact with a user's skin.

In one aspect, the physiological sensors 1322 may include a heart rate monitor or pulse sensor, e.g., where heart rate is optically detected from an artery, such as the radial artery. In one embodiment, the garment 1310 may be configured such that a module 1320 is positioned on a user's wrist, where a physiological sensor 1322 of the module 1320 is secured over the user's radial artery or other blood vessel. Secure connection and placement of a pulse sensor over the radial artery or other blood vessel facilitates measurement of heart rate, pulse oxygen, and the like. It will be understood that this configuration is provided by way of example only, and that other sensors, sensor positions, and monitoring techniques may also or instead be employed without departing from the scope of this disclosure.

In some embodiments, heart rate data may be acquired using an optical sensor coupled with one or more light emitting diodes (LEDs), all in contact with the user 1301. To facilitate optical sensing, the garment 1310 may be designed to maintain a physiological sensor 1322 in secure, continual contact with the skin, and reduce interference of outside light with optical sensing by the physiological sensor 1322.

Thus, certain embodiments include one or more physiological sensors 1322 configured to provide continuous measurements of heart rate using photoplethysmography or the like. The physiological sensor 1322 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 1322 may also or instead provide at least one of continuous motion detection, environmental temperature sensing, electrodermal activity (EDA) sensing, galvanic skin response (GSR) sensing, and the like.

The system 1300 may include different types of modules 1320. For example, a number of different modules 1320 may each provide a particular function. Thus, the garment 1310 may house one or more of a temperature module, a heart rate/PPG module, a muscle oxygen saturation module, a haptic module, a wireless communication module, or combinations thereof, any of which may be integrated into a single module 1320 or deployed in separate modules 1320 that can communicate with one another. Some measurements such as temperature, motion, optical heart rate detection, and the like, may have preferred or fixed locations, and pockets or fixtures within the garment 1310 may be adapted to receive specific types of modules 1320 at specific locations within the garment 1310. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 1300 may include two or more modules 1320 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 1324 may be any as described herein, for example including any of the features of the network interface 1304 described above. The communications interface 1324 may be a separate device that provides the ability for the modules 1320 to communicate with one another and/or with other components of the system 1300), or there may be a central module that communicates with other modules 1320 (or with another component of the system 1300). It will be understood that communications may usefully be secured using any suitable encryption technology in order to ensure privacy and security of user data. This may, for example, include encryption for local (wired or wireless) communications among the modules 1320 and/or controller 1330 within the garment 1310. This may also or instead include encryption for remote communications to a server and other remote resources. In one aspect, the garment 1310 and/or controller 1330 may provide a cryptographic infrastructure for securing local communications, e.g., by managing public/private key pairs for use in asymmetric encryption, authentication, digital signatures, and so forth. The keys for this infrastructure may also or instead be managed by an external, trusted third party.

The controller 1330 may be configured, e.g., by computer executable code or the like, to determine a location of the module 1320. This may be based on contextual measurements such as accelerometer data from the module 1320, which may be analyzed by a machine learning model or the like to infer a body position. In another aspect, this may be based on other signals from the module 1320. For example, signals from sensors such as photodiodes, temperature sensors, resistors, capacitors, and the like may be used alone or in combination to infer a body position. In another aspect, the location may be determined based on a proximity of a module 1320 to a proximity sensor, RFID tag, or the like at or near one of the designated areas 1312 of the garment 1310. Based on the location, the controller 1330 may adapt operation of the module 1320 for location-specific operation. This may include selecting filters, processing models, physiological signal detections, and the like. It will be understood that operations of the controller 1330, which may be any controller, microcontroller, microprocessor, or other processing circuitry, or the like, may be performed in cooperation with another component of the system 1300 such as the processor 1340 described herein, one or more of the modules 1320, or another computing device. It will also be understood that the controller 1330 may be located on a local component of the system 1300 (e.g., on the garment 1310, in a module 1320, and so on) or as part of a remote processing facility 1350, or some combination of these. Thus, in an aspect, a controller 1330 is included in at least one of the plurality of modules 1320. And, in another aspect, the controller 1330 is a separate component of the garment 1310, and serves to integrate functions of the various modules 1320 connected thereto. The controller 1330 may also or instead be remote relative to each of the plurality of modules 1320, or some combination of these.

Location detection (i.e., of the modules 1320 and/or physiological sensors 1322) may also usefully be recorded and used in a number of ways by a human user and/or by the system 1300. For example, a detected location may be stored, along with the corresponding garment, so that a user can retrieve a placement history and replace the module 1320 to a previous location for a particular garment as desired. In another aspect, the detected location may be used by the system 1300 to analyze data and make garment specific recommendations. For example, the system 1300 may evaluate the quality of a signal, e.g., using any conventional metrics such as signal-to-noise ratio, or using quality metrics more specific to physiological signals such as correlation to an expected signal or pulse shape, consistency with a rate or magnitude typical for a sensor, pulse-to-pulse consistency for a particular user, or any other measure of signal quality using statics, machine learning, digital signal processing techniques, or the like. A quality metric, however derived, may be used in turn to recommend specific placements of a module 1320 on a garment 1310 for a user, or to recommend a particular garment 1310 for the user. Thus, for example, after acquiring data over a range of garments and activities, the system 1300 may generate a user-actionable recommendation such as, “It appears that when you are jogging, the most accurate heart rate signals can be obtained when you are wearing an XL shirt model number xxxxxx. You may wish to wear this shirt for active workouts, and you may wish to purchase more of this type of shirt for regular use.” As another example, the user-actionable recommendation may suggest: “It appears that one of your modules is not obtaining accurate temperature readings when located on your sleeve elastic band. You may wish to try a different location for this module, or to try a different garment.” More generally, data quality may be measured for a number of different modules at different locations in different garments during different activities, and this data may be used to generate customized recommendations for a user on a per-garment and per-location basis. These recommendations may also be tailored to specific activity types where this data is accurately recorded by the system 1300, either from user input, automatic detection, or some combination of these.

The controller 1330 may be configured to control one or more of (i) sensing performed by a physiological sensor 1322 of the module 1320 and (ii) processing by the module 1320 of the data received from a physiological sensor 1322. That is, in certain aspects, the combination of sensors in the module 1320 may vary based on where it is intended to be located on a garment 1310. In another aspect, processing of data from a module 1320 may vary based on where it is located on a garment 1310. In this latter aspect, a processing resource such as the controller 1330 or some other local or remote processing resource coupled to the module 1320 may detect the location and adapt processing of data from the module 1320 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 1310, and then use this detected location to select a suitable model for activity recognition. This technique may similarly be applied to calibration models, physiological signals processing models, and the like, or to otherwise adapt processing of signals from a module 1320 based on the location of the module 1320.

Determining the location of a module 1320 may include receiving a sensed location for the module 1320. The sensed location may be provided by a proximity detection circuit such as a near-field-communication (NFC) tag, an (active or passive) RFID tag, a capacitance sensor, a magnetic sensor, an electrical contact, a mechanical contact, and the like. Any corresponding hardware for such proximity detections may be disposed on the module 1320 and the garment 1310 for communication therebetween to detect location when appropriate. For example, in one aspect, an NFC tag may be disposed on or within the garment 1310, and the module may include an NFC tag sensor that can detect the tag and read any location-specific information therefrom. Proximity detection may also or instead be performed using capacitively detected contact, electromagnetically detected proximity, mechanical contact, electrical coupling, and the like. In this manner, a garment 1310 may provide information to an installed module 1320 to inform the module 1320, among other things, where the module 1320 is located, or vice-versa.

Thus, communication between a module 1320 and the garment 1310 (or a processor of the garment 1310) may be used to determine the location of a module 1320 on the garment 1310. Communication of location information may be enabled using active techniques, passive techniques, or a combination thereof. For example, a thin, flexible, cheap, washable NFC tag may be sewn into the garment 1310 in various locations where a module 1320 may be placed. When a module 1320 is placed in the garment 1310, the module 1320 may query an adjacent NFC tag to determine its location. Furthermore, the NFC technique or other similar techniques may provide other information to the module 1320, including details about the garment 1310 such as the size, whether it is a gender specific piece, the manufacturer information, model or serial number of the garment, stock keeping unit (SKU), and more. Similarly, the tag may encode a unique identifier for the garment 1310 that can be used to obtain other relevant information using an online resource. The module 1320 may also or instead advertise information about itself to the garment 1310 so that the garment 1310 can synchronize processing with other modules 1320, synchronize communication among modules 1320, control or condition signals from the module 1320, and so forth. The module 1320 can then configure itself within the context of the current garment 1310 and associated modules 1320, and/or to perform certain types of monitoring or data processing.

Determining the location of a module 1320 may also or instead be based, at least in part, on an interpretation of the data received from a physiological sensor 1322 of the module 1320. By way of example, movement of a module 1320 as detected by a sensor may provide information that can be used to predict a position on or within the garment 1310. Also or instead, the type of data that is being received from a module 1320 may indicate where the module 1320 is located on the garment 1310. For example, locations may produce unique signatures of acceleration, gyroscope activity, capacitive data, optical data, temperature data, and the like, depending on where the module 1320 is located, and this data may be fused and analyzed in any suitable manner to obtain a location prediction.

According to the foregoing, determining the location of a module 1320 may also or instead include receiving explicit input from the user 1301, which may identify one of the designated areas on the garment 1310, or a general area of the body (e.g., left wrist, right ankle, and so forth). Because the location of the module 1320 relative to the garment 1310 may be determined from an analysis of a plurality of data sources, the system 1300 may include a component (e.g., the processor 1340) that is configured to reconcile one or more potential sources of location of information based on expected reliability, measured quality of data, express user input, and so forth. A prediction confidence may also usefully be generated in this context, which may be used, for example, to determine whether a user should be queried for more specific location information. More generally, any of the foregoing techniques may be used along or in combination, along with a failsafe measure the requests user input when location cannot confidently be predicted. Also or instead, a user may explicitly specify a prediction preemptively, or as an override to an automatically generated prediction.

Once determined using any of the techniques above, the location of a module 1320 may be transmitted for storage and analysis to a remote processing facility 1350, a database 1360, or the like. That is, in addition to the module 1320 using this information locally to configure itself for the location in which it is worn, the module 1320 may communicate this information to other modules 1320, peripherals, or the cloud. Processing this information in the cloud may help an organization determine if a module 1320 has ever been installed on a garment 1310, which locations are most used, and how modules 1320 perform differently in different locations. These analytics may be useful for many purposes, and may, for example, be used to improve the design or use of modules 1320 and garments 1310, either for a population, for a user type, or for a particular user.

As stated above, the system 1300 may further include a processor 1340 and a memory 1342. In general, the memory 1342 may bear computer executable code configured to be executed by the processor 1340 to perform processing of the data received from one or more modules 1320. One or more of the processor 1340 and the memory 1342 may be located on a local component of the system 1300 (e.g., the garment 1310, a module 1320, the controller 1330, and the like) or as part of a remote processing facility 1350 or the like as shown in the figure. Thus, in an aspect, one or more of the processor 1340 and the memory 1342 is included on at least one of the plurality of modules 1320. In this manner, processing may be performed on a central module, or on each module 1320 independently. In another aspect, one or more of the processor 1340 and the memory 1342 is remote relative to each of the plurality of modules 1320. For example, processing may be performed on a connected peripheral device such as smart phone, laptop, local computer, or cloud resource.

The memory 1342 may store one or more algorithms, models, and supporting data (e.g., parameters, calibration results, user selections, and so forth) and the like for transforming data received from a physiological sensor 1322 of the module 1320. In this manner, suitable models, algorithms, tuning parameters, and the like may be selected for use in transforming the data based on the location of the module 1320 as determined by the controller 1330 and/or processor 1340 as described herein. By way of example, algorithms that convert data from an accelerometer in a module 1320 into a count of a user's steps may be different depending on whether the module 1320 is worn on the user's wrist or on the user's waist band. Similarly, the intensity of an LED and corresponding sensitivity of a photodetector may be different for a PPG device placed on the wrist or the thigh. Thus, the module 1320 may self-configure for a location by controlling one or more of sensor types, sensor parameters, processing models, and so forth based on a detected location for the module 1320.

Selection of an algorithm may also or instead include an analysis of one or more of the sensor data, metadata, and the like. By way of example, an algorithm may be selected at least in part based on metadata received from one of the module 1320 and the garment 1310. This metadata may be derived from communication between the module 1320 and the garment 1310—e.g., between a tag and tag reader for exchanging information therebetween. For example, the garment 1310 may include, e.g., stored in a tag such as an NFC tag or other wirelessly readable data source, garment-specific metadata that is readable by or otherwise transmittable to one or more of the plurality of modules 1320, the controller 1330, and the processor 1340. Such garment-specific metadata may include at least one of a type of garment 1310, a size of the garment 1310, garment dimensions, a gender configuration of the garment 1310, a manufacturer, a model number, a serial number, a SKU, a material, fit information, and so on. In one aspect, this information may be provided with one or more of the location identification tags described herein. In another aspect, the garment 1310 may include an additional tag at a suitable location (e.g., near or accessible to a processor or controller) that provides garment-specific information while other tags provide location-specific information.

The metadata may also or instead include at least one of a gender of the user 1301, a weight of the user 1301, a height of the user 1301, an age of the user 1301, metadata associated with the garment 1310 (e.g., the garment size, type, material, etc.), and the like. The metadata may be derived, at least in part, from user-provided input, or otherwise from information derived from the user 1301 such as a user's account information as a participant in the system 1300. By way of example, a processing algorithm may be selected depending on the material of the garment 1310 as communicated by its serial or model number in an identification tag, the physiology of the user 1301 as implied by the garment size, and so on. The metadata may also or instead be used to verify the authenticity of the garment 1310, and otherwise control access to the garment 1310 and/or modules 1320 coupled to the garment 1310. In one aspect, metadata (e.g., size, material) may be encoded directly into the garment metadata. In another aspect, the garment 1310 may publish a unique identifier that can be used to retrieve related information from a manufacturer or other data source. This latter approach advantageously permits correlation of garment-specific data with other user-specific data such as height, weight, body composition, and so forth.

Simply knowing a priori where a module 1320 is positioned may allow for the use of algorithms that have been developed to perform optimally in that particular location. This can relieve a significant computational burden otherwise borne by the module 1320 to analytically evaluate location based on available signals. Other information may also or instead be used to select an optimal algorithm. For example, based on the gender or dimensions of a garment, the algorithm may employ different models or different model parameters.

The processor 1340 may be configured to assess the quality of the data received from a physiological sensor 1322 of the module 1320. For example, the processor 1340 may be configured to provide, based on the quality of the data, a recommendation regarding at least one of the location of a module 1320 and an aspect of the garment 1310 (e.g., size, fit, material, and so on). For example, the processor 1340 may be configured to detect when the garment does not properly fit the wearer for acquisition of physiological data, for example, by detecting when a module is moving (e.g., from accelerometer data) but data quality is poor or absent for a sensed physiological signal. In general, the garment 1310 may store its own identifier and/or metadata, e.g., as described herein, or garment identification data may be stored in tags, e.g., at designated areas 1312 of the garment 1310. The processor 1340 may be configured to use this garment identification information and/or metadata to provide a recommendation regarding a different garment 1310 for the user 1301, or for an adjustment to the current garment 1310. For example, if a particular garment 1310 seems to result in low-quality data, the user 1301 could be encouraged to select an alternative size, or to make some other adjustment. Moreover, data on how many times a garment 1310 is used may be gathered and used to inform business decisions, for example, which garments 1310 provide the highest-quality data, and which garments 1310 are most preferred by users 1301.

The system 1300 may further include a database 1360, which may be located remotely and in communication with the system 1300 via the data network 1302. The database 1360 may store data related to the system 1300 such as any discussed herein—e.g., sensed data, processed data, transformed data, metadata, physiological signal processing models and algorithms, personal activity history, and the like. The system 1300 may further include one or more servers 1370 that host data, provide a user interface, process data, and so forth in order to facilitate use of the modules 1320 and garments 1310 as described herein.

It will be appreciated that the garment 1310, modules 1320, and accompanying garment infrastructure and remote networking/processing resources, may advantageously be used in combination to improve physiological monitoring and achieve modes of monitoring not previously available.

One or more of the devices and systems described herein may include circuitry for both wireless charging and wireless data transmission, e.g., where the corresponding circuits can operate independently from one another, and where the corresponding antennae are located proximal to one another (for instance, the circuitry for wireless charging and the circuitry for wireless data transmission may include separate coils disposed substantially along the same plane, or otherwise in relative close proximity in a device or system). In such aspects, one or more measures may be taken so that a wireless data transfer process does not interfere with a wireless power transfer process, more specifically by coupling the data circuitry into the electromagnetic field for the wireless power transfer in a manner that alters the resonant frequency or otherwise destructively interferes with power transfer, thereby decreasing efficiency when charging a device. For example, a switch may be included to disable circuitry for data transmission when certain wireless charging activity is present, thereby allowing for relatively unimpeded and efficient wireless charging of a device. The switch may also be operable to enable operation of data transmission circuitry when certain wireless charging activity is not present.

Thus, for example, in the context of a physiological monitor, such as any of those described herein, the physiological monitor may include both a wireless power receiver (or similar) and a wireless data tag reader (or similar). In general, these sub-systems may conform to one or more Near Field Communication (NFC) specifications for protocols and physical architectures, or any other standards suitable for wireless power and data transmission. The power circuitry may be used, e.g., to charge a battery on the physiological monitor so that the device can be recharged without physically connecting to a power source. The data circuitry may be used, e.g., as a wireless data tag reader or the like to read data from nearby data sources such as identification tags in user apparel and the like. In general, the physiological monitor may include separate circuitry (separate coils) for these wireless power and data systems, such as separate processing circuitry and/or separate antennae. The antennae may be disposed substantially along the same plane of the physiological monitor (e.g., with one coil disposed substantially inside or adjacent to the other). In one aspect, the antennae may be in parallel planes, however, it will be noted that distance tolerances for NFC standard devices are relatively small, and the physically housing for these antennae will preferably enforce an identical or substantially identical distance for both antennae in such architectures. In this context, the positions of the antennae may be as close to parallel as possible within reasonable manufacturing tolerances, or as close to parallel as possible when disposed on two different layers of a shared printed circuit board, or preferably, when disposed on a single layer of a shared printed circuit board. The physiological monitor may further include a switch (e.g., a radio frequency (RF) switch or the like) in-line with the coil for the wireless data tag reader to disable the wireless data tag reader when power is being received to mitigate any effects on the efficiency of the wireless power transfer process. In particular, the switch may be configured to open when power is being received, and may be configured to close when the physiological monitor is looking for data tag to read.

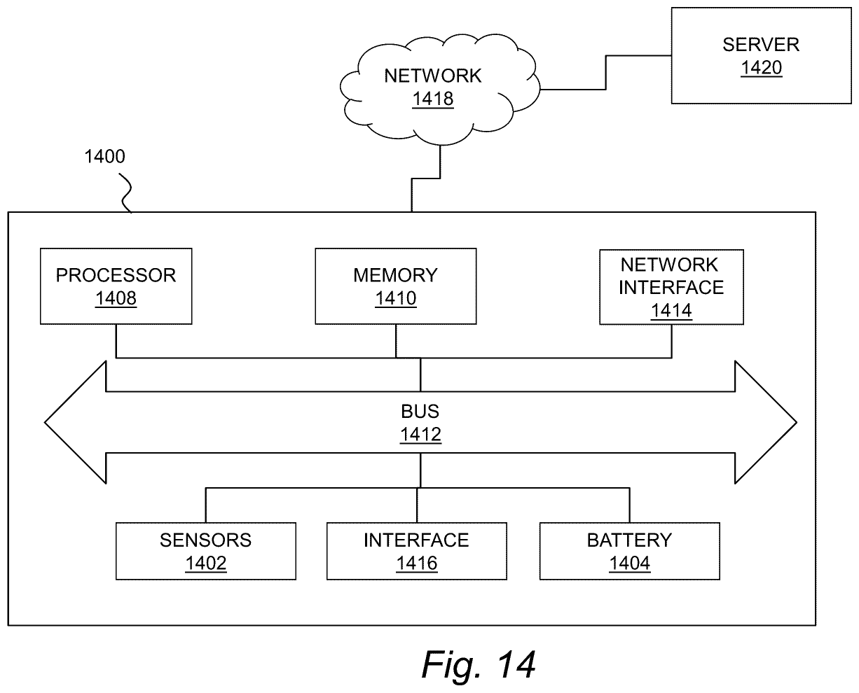

FIG. 14 is a block diagram of a computing device 1400. The computing device 1400 may, for example, be a device used for continuous physiological monitoring, or any other device supporting a physiological monitor in the systems and methods described herein. The device may also or instead be any of the local computing devices described herein, such as a desktop computer, laptop computer, smart phone. The device may also or instead be any of the remote computing resources described herein, such as a web server, a cloud database, a file server, an application server, or any other remote resource or the like. While described as a physical device, it will be understood that the exemplary computing device 1400 may also or instead be realized as a virtual computing device such as a virtual machine executing a web server or other remote resource in a cloud computing platform. In general, the device 1400 may include one or more sensors 1402, a battery 1404, storage, a processor 1408, memory 1410, a network interface 1414, and a user interface 1416, or virtual instances of one or more of the foregoing.

The sensors 1402 may include any sensor or combination of sensors suitable for heart rate monitoring as contemplated herein, as well as sensors 1402 for detecting calorie burn, position (e.g., through a Global Positioning System or the like), motion, activity and so forth. In one aspect, this may include optical sensing systems including LEDs or other light sources, along with photodiodes or other light sensors, that can be used in combination for photoplethysmography measurements of heart rate, pulse oximetry measurements, and other physiological monitoring.

The sensors 1402 may also or instead include one or more sensors for activity measurement. In some embodiments, the system may include one or more multi-axes accelerometers and/or gyroscope to provide a measurement of activity. In some embodiments, the accelerometer may further be used to filter a signal from the optical sensor for measuring heart rate and to provide a more accurate measurement of the heart rate. In some embodiments, the wearable system may include a multi-axis accelerometer to measure motion and calculate distance. Motion sensors may be used, for example, to classify or categorize activity, such as walking, running, performing another sport, standing, sitting or lying down. The sensors 1402 may, for example, include a thermometer for monitoring the user's body or skin temperature. In one embodiment, the sensors 1402 may be used to recognize sleep based on a temperature drop, Galvanic Skin Response data, lack of movement or activity according to data collected by the accelerometer, reduced heart rate as measured by the heart rate monitor, and so forth. The body temperature, in conjunction with heart rate monitoring and motion, may be used, e.g., to interpret whether a user is sleeping or just resting, as well as how well an individual is sleeping. The body temperature, motion, and other sensed data may also be used to determine whether the user is exercising, and to categorize and/or analyze activities as described in greater detail below. In another aspect, the sensors 1402 may include one or more contact sensors, such as a capacitive touch sensor or resistive touch sensor, for detecting placement of a physiological monitor for use on a user. More generally, the sensors 1402 may include any sensor or combination of sensors suitable for monitoring geographic location, physiological state, exertion, movement, and so forth in any manner useful for physiological monitoring as contemplated herein.

The battery 1404 may include one or more batteries configured to allow continuous wear and usage of the wearable system. In one embodiment, the wearable system may include two or more batteries, such as a removable battery that may be removed and recharged using a charger, along with an integral battery that maintains operation of the device 1400 while the main battery charges. In another aspect, the battery 1404 may include a wireless rechargeable battery that can be recharged using a short range or long range wireless recharging system.

The processor 1408 may include any microprocessor, microcontroller, signal processor or other processor or combination of processors and other processing circuitry suitable for performing the processing steps described herein. In general, the processor 1408 may be configured by computer executable code stored in the memory 1410 to provide activity recognition and other physiological monitoring functions described herein.

In general the memory 1410 may include one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, optical disks, USB flash drives), and the like. In one aspect, the memory 1410 may include a computer system memory or random access memory, such as DRAM, SRAM, EDO RAM, and the like. The memory 1410 may include other types of memory as well, or combinations thereof, as well as virtual instances of memory, e.g., where the device is a virtual device. In general, the memory 1410 may store computer readable and computer-executable instructions or software for implementing methods and systems described herein. The memory 1410 may also or instead store physiological data, user data, or other data useful for operation of a physiological monitor or other device described herein, such as data collected by sensors 1402 during operation of the device 1400.

The network interface 1414 may be configured to wirelessly communicate data to a server 1420, e.g., through an external network 1418 such as any public network, private network, or other data network described herein, or any combination of the foregoing including, e.g., local area networks, the Internet, cellular data networks, and so forth. Where the device is a physiological monitoring device, the network interface 1414 may be used, e.g., to transmit raw or processed sensor data stored on the device 1400 to the server 1420, as well as to receive updates, receive configuration information, and otherwise communicate with remote resources and the user to support operation of the device. More generally, the network interface 1414 may include any interface configured to connect with one or more networks, for example, a Local Area Network (LAN), a Wide Area Network (WAN), the Internet, or a cellular data network through a variety of connections including, but not limited to, standard telephone lines, LAN or WAN links (for example, 802.11, T1, T3, 56 kb, X.25), broadband connections (for example, ISDN, Frame Relay, ATM), wireless connections, or some combination of any or all of the above. The network interface 1412 may include a built-in network adapter, network interface card, PCMCIA network card, card bus network adapter, wireless network adapter, USB network adapter, modem or any other device suitable for interfacing the computing device 1400 to any type of network capable of communication and performing the operations described herein.

The user interface 1416 may include any components suitable for supporting interaction with a user. This may, for example, include a keypad, display, buzzer, speaker, light emitting diodes, and any other components for receiving input from, or providing output to, a user. In one aspect, the device 1400 may be configured to receive tactile input, such as by responding to sequences of taps on a surface of the device to change operating states, display information and so forth. The user interface 1416 may also or instead include a graphical user interface rendered on a display for graphical user interaction with programs executing on the processor 1408 and other content rendered by a physical display of device 1400.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “continuous,” as used herein in connection with heart rate data, refers to the acquisition of heart rate data at a sufficient frequency to enable detection of individual heartbeats, and also refers to the collection of heart rate data over extended periods such as a day or more (including acquisition throughout the day and night). More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. At the same time, continuous monitoring is not intended to exclude ordinary data acquisition interruptions such as temporary displacement of monitoring hardware due to sudden movements, changes in external lighting, loss of electrical power, physical manipulation or adjustment by a wearer, physical displacement of monitoring hardware due to external forces, and so forth. It will also be noted that heart rate data or a monitored heart rate, in this context, may more generally refer to raw sensor data, or processed data therefrom such as heart rate data, signal peak data, heart rate variability data, or any other physiological or digital signal suitable for recovering heart rate information as contemplated herein, and that heart rate data may generally be captured over some historical period that can be subsequently correlated to various metrics such as sleep states, activity recognition, resting heart rate, maximum heart rate, and so forth.

The term “computer-readable medium,” as used herein, refers to a non-transitory storage hardware, non-transitory storage device or non-transitory computer system memory that may be accessed by a controller, a microcontroller, a microprocessor, a computational system, or a module of a computational system to encode thereon computer-executable instructions or software programs. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

The above illustrative examples of various aspects and implementations refer to prediction models or machine learning models. The skilled person will appreciate that, even if not expressly stated, a prediction model or machine learning model can be trained using standard training approaches as known in the art. For example, a standard approach for training a prediction model comprises obtaining relevant training data (e.g., using known data sources, databases, or data sets) and performing cross-validation to train the prediction model on the training data. Typically, cross-validation involves splitting the training data into K-folds (approximately equal partitions or sets of the training data) and withholding a single fold as a test set and, one by one, using one of the remaining folds as a validation set and the remaining K−2 folds as a training set. The model is then repeatedly trained on the training set using different model hyperparameters and the performance validated on the validation set. Once the best performing hyperparameters are obtained, the model trained according to the best hyperparameters are evaluated on the test set. For training classification models, the cross-validation strategy can be stratified such that the proportion of training instances within each category or class is approximately the same across each fold. Model hyperparameters can be selected using any suitable approach such as grid search or randomized search. Model performance can be estimated using any suitable performance measure and is dependent on the type of model being trained (e.g., mean square error for regression, binary cross entropy for classification, ranking loss for ranking, etc.).

### Results

TABLE V

| | | | | % Err < 10 |
|---|---|---|---|---|
| Source | Measure | MAE (mmHg) | ME (mmHg) | mmHg |
| Study 1 | SBP | 7.61 (±5.73) | 0.10 (±9.53) | 70.06% |
| Study 1 + | SBP | 8.20 (±6.38) | 0.15 (±10.39) | 67.40% |
| 2 | SBP (dem. | 848 (±6.95) | −0.37 (±10.96) | 66.57% |
| | only) | | | |
| Study 1 | DBP | 5.95 (±4.80) | −0.64 (±7.62) | 81.44% |
| Study 1 + | DBP | 6.73 (±4.93) | 0.16 (±8.34) | 77.44% |
| 2 | DBP (dem. | 7.10 (±5.17) | 0.51 (±8.78) | 73.71% |
| | Only) | | | |

Table V shows results of utilizing the systems and methods of the present disclosure for estimation of systolic blood pressure (SBP) and diastolic blood pressure (DBP). The results were obtained on datasets of two studies: Study 1 comprising static, dynamic, and sleep data for approximately 350 studies, and Study 2 comprising static, dynamic, sleep data, and demography data for approximately 310 studies. The results are obtained with a 5-fold cross validation approach with no user (even if participated in multiple studies) being a part of train and test data at the same time. The trained models produce an estimate for each segment/sample of each study. Since each study comprises multiple samples/segments, obtaining a final mean estimate for each study is obtained by taking the mean value of corresponding estimates. The model performance was computed based on the final estimates only.

TABLE VI

Actual

| | | Hypertensiv | Normotensive |
|---|---|---|---|
| Predicted | Normotensive | 0.19 | 0.77 |
| | Hypertensive | 0.81 | 0.23 |

TABLE VII

Actual

| | | Hypertensiv | Normotensive |
|---|---|---|---|
| Predicted | Normotensive | 0.26 | 0.74 |
| | Hypertensive | 0.74 | 0.26 |

Tables VI and VII show classification results of using the systems and methods of the present disclosure to estimate systolic hypertension classification (Table VI) and diastolic hypertension classification (Table VII). The results are obtained on the Study 1+2 data described above with the same 5-fold cross validation approach being used. For systolic hypertension classification, an accuracy of 0.78 was achieved with an AUC of 0.82. For diastolic hypertension classification, an accuracy of 0.74 was achieved with an AUC of 0.81.

1. A method for baseline blood pressure estimation of a user of a wearable physiological monitor, the method comprising:
identifying a first segment of pulse data related to cardiac activity of the user during a first portion of a sleep session, wherein the first segment of pulse data is obtained by the wearable physiological monitor;
determining, from the first segment of pulse data, a first resting heart rate value of the user during the first portion of the sleep session;
identifying a machine learning model trained to receive as input one or more features including an input resting heart rate value obtained during a first time period and predict an indicator of blood pressure during a second time period; and
providing the first resting heart rate value to the machine learning model to obtain a first indicator of baseline blood pressure for the user.

2. The method of claim 1, wherein the first time period is during nighttime of the sleep session.

3. The method of claim 1, wherein the second time period is during daytime on a day following the sleep session.

4. The method of claim 1, further comprising:
extracting, from the first segment of pulse data, one or more static features that characterize an average pulse morphology during the first portion of the sleep session.

5. The method of claim 4, further comprising providing the one or more static features as input to the machine learning model to obtain the first indicator of baseline blood pressure.

6. The method of claim 4, wherein the one or more static features include any one or more of: an average pulse width value; an average maximum acceleration value; an average maximum derivative value; an average time to maximum derivative or acceleration value; an average area under the curve value; an average area without detrending value; an average time between systolic and diastolic peaks value; one or more latent features.

7. The method of claim 1, further comprising:
extracting, from the first segment of pulse data, one or more dynamic features that characterize temporal variation in pulse morphology during the first portion of the sleep session.

8. The method of claim 7, further comprising providing the one or more dynamic features to the machine learning model to obtain the first indicator of baseline blood pressure.

9. The method of claim 7, wherein the one or more dynamic features are generated from a plurality of morphology features extracted from each pulse in the first segment of pulse data.

10. The method of claim 9, wherein the plurality of morphology features extracted for a pulse of the first segment of pulse data include any two or more of: a pulse width value; a maximum acceleration value; a maximum derivative value; a time to maximum value; a time to maximum acceleration value; an area under the curve value; an area without detrending value; a time between systolic and diastolic peaks value; an instantaneous pulse rate value; a pulse amplitude value; one or more latent features; a notch metric value indicative of an extent of a dicrotic notch.

11. The method of claim 10, wherein the one or more latent features are determined using an encoder-decoder neural network.

12. The method of claim 10, wherein the notch metric value is extracted using an encoder-decoder neural network.

13. The method of claim 12, wherein the encoder-decoder neural network is a variational autoencoder.

14. The method of claim 12, wherein the encoder-decoder neural network is trained on a dataset of synthetic pulses and annotated real pulses, and wherein a reconstruction loss value extracted using the encoder-decoder neural network is indicative of a quality of the pulse.

15. The method of claim 9, further comprising:
providing the plurality of morphology features to a Recurrent Neural Network (RNN) to generate the one or more dynamic features, wherein the RNN is trained to output dynamic features from pulse morphology features provided as input.

16. The method of claim 15, wherein the RNN is one of a stacked Long Short Term Memory (LSTM) network or a Gated Recurrent Unit (GRU) network.

17. The method of claim 1, further comprising providing one or more features that characterize demographic data of the user to the machine learning model to obtain the first indicator of baseline blood pressure.

18. The method of claim 1, further comprising:
identifying sleep data that characterizes the first portion of the sleep session in relation to the sleep session; and
providing the sleep data as an additional input to the machine learning model to obtain the first indicator of baseline blood pressure.

19. The method of claim 18, wherein the sleep data comprises sleep onset data indicative of a start time of the first portion of the sleep session in relation to a start of the sleep session.

20. The method of claim 18, wherein the sleep data comprises sleep stage data.

21-101. (canceled)
